(19) United States
(12) Patent Application Publication
(10) Pub. No.: US 2025/0325211 A1
(43) Pub. Date: Oct. 23, 2025

(54) Devices For Chronic Clinical Grade Electrophysiology

(71) Applicant: Arizona Board of Regents, Tucson, AZ (US)

(72) Inventors: Philipp Gutruf, Tucson, AZ (US); Tucker Stuart, Tucson, AZ (US); Dylan McGuire, Tucson, AZ (US)

(21) Appl. No.: 19/187,008

(22) Filed: Apr. 23, 2025

Related U.S. Application Data
(60) Provisional application No. 63/637,651, filed on Apr. 23, 2024.

(51) Int. Cl. A61B 5/263; A61B 5/00; A61B 5/053; A61B 5/085; A61B 5/28

(52) U.S. Cl. CPC A61B 5/263; A61B 5/053; A61B 5/086; A61B 5/28; A61B 5/6801; A61B 2560/0219; A61B 2560/0468; A61B 2562/125; A61B 2562/227

(57) ABSTRACT

The present disclosure provides biosymbiotic systems and devices for continuous monitoring of electrophysiological biosignals. The systems and devices described herein include carbon-doped filament deposition modeling (FDM) printed dry electrodes that overcome impedance degradation by seamless integration into textile and wearable biosymbiotic platforms, allowing for high fidelity operation over indefinite timescales. The systems and devices also include at-distance wirelessly powered wearable electronics. The systems and devices described herein can be used to monitor ECG/EIP during work, activity, and sleep and BioZ recordings documenting gains in forearm training over weeks.

### CROSS REFERENCE TO RELATED APPLICATIONS

The present application claims the benefit of U.S. Provisional Application Ser. No. 63/637,651, filed Apr. 23, 2024, which is hereby incorporated by reference in its entirety.

### TECHNICAL FIELD

The present disclosure relates to devices for chronic, clinical-grade electrophysiology, including wearable, form fitting devices and textile-integrated devices for chronic, clinical-grade electrophysiology.

### BACKGROUND

The electrocardiogram (ECG) provides critical diagnostic information related to cardiovascular disease. Clinical standards in portable ECG rely on devices such as the Holter monitor, a device with large mass and limited battery life, paired with adhesive-backed Ag/AgCl gel electrodes that are known to rapidly degrade. Fundamental disadvantages of adhesively mounted electrophysiology devices relate to epidermal turnover at the adhesive-skin interface and decreased signal quality and user comfort. These limitations extend to on-body chronic measurement of Bioimpedance (BioZ), a modality that uses alternating current (AC) injection to evaluate physiological changes in cell architecture or fluid. Wearable BioZ devices take on many forms, examples being Electrical Impedance Pneumography (EIP), which evaluates changes in tidal flow, pleural fluid content, and respiratory muscle activation, crucial metrics for evaluation of respiratory diseases such as asthma, COVID-19, and COPD. Only a handful of research teams have explored textile-integrated strategies for continuous monitoring of this parameter, and systems reporting chronic-use capabilities lack appropriate skin-electrode interface infrastructure. Additionally, the modality Electrical Impedance Myography (EIM) represents a useful tool in quantifying architectural changes in the cell structure caused by sarcomere overlap at contraction or degenerative neuromuscular diseases such as various myopathies and sarcopenia related to aging. Chronic measurement typically is limited to snapshots relying on daily measurements of latent impedance with newly applied gel electrodes, and wearable systems currently do not attempt uninterrupted data streaming over indefinite timescales.

Recent research addresses singular components of these drawbacks through dry or semi-dry electrodes made of a combination of gold, carbon, titanium, stainless steel, or organic polymer composites capacitively coupled to skin which show promise in long-term stability for chronic signal acquisition. Innovations such as epidermal electrodes, microneedle solutions, and soft electrode systems, however, still rely on adhesive backings which face the same chronic-use constraints from epidermal turnover. Dry electrode systems that address the adhesive issue typically are integrated into a t-shirt platform with woven conductive threads, PEDOT:PSS ink staining, or tight-fitting chest straps. These electrode interfaces offer promising signal fidelity but raise issues related to breathability and comfort and rely on electrochemical energy sources and bulky electronics that limit operational time to days. Combined, these technical limitations result in electrophysiological monitoring solutions that do not provide the desired 24/7 high-fidelity monitoring required to catch rare but critical cardiovascular defects required for the diagnosis of conditions such as intermittent atrial fibrillation and other arrythmias, progression of acute illnesses defined by changes in respiratory rate and depth, and progression of neuromuscular conditions and atrophy that negatively affect muscular cell density and in turn, impedance readings.

### BRIEF DESCRIPTION OF THE DRAWINGS

Features and advantages of various embodiments of the claimed subject matter will become apparent as the following Detailed Description proceeds, and upon reference to the Drawings, wherein like numerals designate like parts, and in which:

FIGS. 1A-1B illustrate an overview of the electrophysiological devices of embodiments the present disclosure where FIG. 1A illustrates electrodes integrated with a textile and TPU-based electrophysiological biosymbiotic wearable platforms, highlighting continuous, around-the-clock data collection; and FIG. 1B illustrates various diagrams of high-level functional and block diagrams of operating principles of the present disclosure including Electrocardiogram (ECG) and Bioimpedance (BioZ) applications;

FIGS. 1A-2E illustrate example devices and testing of example devices according to embodiments of the present disclosure; where FIG. 2A illustrates details of the electrode integration and connectivity for the textile-integrated devices (left-most panel, upper panel and upper middle panel of FIG. 2A) and the biosymbiotic wearable devices (left-most panel, lower panel and lower middle panel of FIG. 2A) according to various embodiments of the present disclosure; and FIG. 2B illustrates a textile-integrated mechanical hysteresis curve for resistance change with a strain input of 30%, where the shaded area represents standard deviation from the mean response (n=5); FIG. 2C illustrates a biosymbiotic-integrated hysteresis curve for resistance change with a strain input of 30%, where the shaded area represents standard deviation from the mean response (n=5); FIG. 2D illustrates a curve of ultimate strength and failure modes for textile-integrated electrodes of the present disclosure; and FIG. 2E illustrates a curve of ultimate strength and failure modes for biosymbiotic-integrated electrodes of the present disclosure;

FIGS. 3A-3H illustrate electrode characterization and geometric effects on signal quality of the devices of the present disclosure; where FIG. 3A illustrates a schematic of ECG signal acquisition of the devices of the present disclosure integrated into a shirt; FIG. 3B (left) illustrates electrode performance of the devices of the present disclosure by mesh geometry vs. a “gold standard” Ag/AgCl electrode for peak-peak amplitude, and (right) FDM printed electrode geometry signal correlation to simultaneously collected Ag/AgCl ECG signal; FIG. 3C illustrates a schematic of EIP signal acquisition of the devices of the present disclosure integrated into a shirt; FIG. 3D (left) illustrates electrode performance by geometry vs the gold standard Ag/AgCl electrode for tidal breathing peak-peak amplitude, and (right) illustrates electrode performance for large, dense geometry evaluated as change in BioZ amplitude from maximal expiration to maximal inspiration; FIG. 3E illustrates a schematic of EIM testing with waveform overlap of multiple hand dynamometer squeezes at 15 kg with electrodes placed along the brachialis; FIG. 3F (left) illustrates the average change in bioimpedance from rest to over a 5 second 15 kg hand dynamometer squeeze, and (right) illustrates the signal to noise ratio of each electrode type; FIG. 3G illustrates a schematic of motion artifact induction experiments for walking and running; FIG. 3H (left) illustrates root mean square (RMS) error of overlapped waveforms and R\hat{ }2 (signal correlation) to generated idealized line of best fit, and (right) illustrates artifact amplitude and settling time averaged over jump tests;

FIGS. 4A-4D illustrate stability over chronic timescales for electrodes of the present disclosure compared to conventional gel electrodes; where FIG. 4A illustrates simultaneously collected ECG waveforms from electrodes overlaid on Ag/AgCl gel electrodes at 144 hours of continuous wear; FIG. 4B illustrates peak-to-peak amplitude voltage readings of R and S waves for 60 s of ECG signal plotted once per day; FIG. 4C illustrates normalized impedance pneumography waveforms detailed signal quality and fidelity of an Ag/AgCl electrode for tidal breathing and breath holds; and FIG. 4D illustrates trends in skin-electrode contact impedance for biosymbiotic electrodes vs varying brands of commercial gel electrodes. Experiment terminated when gel electrodes lost adhesive strength;

FIGS. 5A-5H illustrate variable use-cases and chronic experimentation; where FIG. 5A illustrates a correlation plot comparing HR calculated from R-R peak intervals in the textile-integrated system against the Polar H10 chest strap; FIG. 5B illustrates a Bland-Altman plot comparing HR calculated from R-R peak intervals in the textile-integrated platform against the Polar H10 chest strap; FIG. 5C illustrates waveform overlap for an example data collection instance where the subject sat motionless for 10 minutes, then ran up and down stairs for another fifteen minutes; FIG. 5D illustrates signal quality and HR plot during continuous monitoring of ECG over a 22-hour data collection period; FIG. 5E illustrates signal quality and HR plot during continuous sleep monitoring for a female subject; FIG. 5F illustrates experimental protocol overview for EIM chronic testing. Inset: example waveform generated during a test; FIG. 5G illustrates training progress during the exercise protocol; and FIG. 5H illustrates EIM trends per day following the strength protocol;

FIGS. 6A-6F illustrate an annotated circuit diagram; where FIG. 6A illustrates example device layout; FIG. 6B illustrates a close-up view of relevant circuit components of FIG. 6A; FIG. 6C a illustrates close-up view of Bluetooth and antenna circuitry of FIG. 6A; FIG. 6D illustrates a close-up view of a battery management circuit of FIG. 6A; FIG. 6E illustrates a close-up view of a rectifier bridge circuit and antenna of FIG. 6A; and FIG. 6F illustrates tables of example components and component values for the circuit components of FIGS. 6A-E;

FIGS. 7A-7C illustrate cross-sectional views of electrode assemblies; where FIG. 7A illustrates a cross sectional view of the electrode assembly for a textile integration, taken along line X-X of the left panel of FIG. 7C; FIG. 7B illustrates a cross sectional view of the electrode assembly for a wearable integration, taken along line XI-XI of the right panel of FIG. 7C; and FIG. 7C illustrates top down views of the electrodes according to embodiments of the present disclosure;

FIG. 8 illustrates textile integration strategies and adhesive strength peel resistance in grams evaluated by ASTM 1876 T-Peel test;

FIGS. 9A-9C illustrate mechanical and electrical hysteresis curves; where FIG. 9A illustrates a curve for biosymbiotic-integrated electrodes; FIG. 9B illustrates a curve for textile-integrated utilizing adhesive+ironing integration methods; and FIG. 9C illustrates textile-integrated electrodes FDM printed directly into textile+ironing;

FIG. 10 illustrates strain experienced from repeating donning and doffing of the textile-integrated platform of the present disclosure;

FIG. 11 illustrates electrode geometry characteristics according to embodiments of the present disclosure, including acronyms and photos;

FIG. 12 illustrates gel electrodes EIP frequency characterization (N=5);

FIG. 13 illustrates line of best-fit and overlaid original waveforms for calculation of RMSE during controlled walking motion-artifact induction, comparing conventional electrode (left and right panels) to the electrode of the present disclosure (middle panel);

FIG. 14 illustrates electrolytic deposition on biosymbiotic electrodes from continuous wear;

FIGS. 15A-15C illustrate textile-integrated electrophysiology device performance vs. a gold standard; where FIG. 15A illustrates a waveform overlay of calculated HR from biosymbiotic electrodes vs Polar H10 calculated values; FIG. 15B illustrates a zoomed in region of significant difference between textile-integrated device and gold standard; and FIG. 15C illustrates a further zoomed in region showing accurate peak detection for waveform overlap mismatch;

FIG. 16 illustrates an example hand dynamometer loading scheme for a day of testing;

FIG. 17 illustrates a battery discharge curve; where the left side indicates the biosymbiotic EIM device of the present disclosure discharging away from a power caster; and the right side indicates timeframes where the device was able to recharge, with less than five minutes away from the power caster;

FIGS. 18A-18I illustrate example fabrication steps of a wearable biosymbiotic electrophysiology system according to the teachings of the present disclosure; where FIG. 18A illustrates FDM printing nonconductive TPU biosymbiotic mesh; FIGS. 18B, C, and D illustrate visualization of assembly of the conductivity elements to an electrode mesh; FIGS. 18E and F illustrate parchment paper overlay for heat-bonding with an iron; FIG. 18G is a photo of the electrode assembly; FIG. 18H illustrates heat bonding of the electrode with the biosymbiotic mesh platform; and FIG. 18I illustrates integration with electronics using the steps illustrated in reference to FIG. 20;

FIGS. 19A-19K illustrate example fabrication steps for the textile-integrated electrophysiology systems according to the teachings of the present disclosure; where: FIG. 19A illustrates FDM printing nonconductive TPU device housing into the textile; FIG. 19B illustrates integrating electrodes via direct printing (top), or fabric adhesive (bottom); FIG. 19C illustrates parchment paper overlay for heat-bonding using an iron; FIG. 19D illustrates creating a through-hole in the textile for electronic integration; FIGS. 19E, F, and G visualization of assembly of the conductivity elements to an electrode in association with the textile; FIG. 19H illustrates parchment paper overlay for heat-bonding using an iron; FIG. 19I is a photo of the assembly; and FIGS. 19J, and K illustrate integration with electronics using the steps illustrated in reference to FIG. 20; and

FIGS. 20A-20I illustrate example steps for electronics fabrication for biosymbiotic and textile-integrated electrophysiology systems according to the teachings of the present disclosure, where; FIG. 20A illustrates disposing FlexPCB/Wire into nonconductive TPU serpentine housing and encapsulating; FIG. 20B illustrates soldering wire to FlexPCB component of electrode, if needed; FIG. 20C illustrates placing the device in a nonconductive TPU housing; FIGS. 20D, and E illustrate beginning to encapsulate using UV curable white flexible resin; FIG. 20F illustrates attaching wire/FlexPCB to biopotential breakout pins G) complete encapsulation H) photographic images of completed biosymbiotic and textile-integrated systems.

Although the following Detailed Description will proceed with reference being made to illustrative embodiments, many alternatives, modifications and variations thereof will be apparent to those skilled in the art.

### DETAILED DESCRIPTION

The present disclosure provides carbon-based Filament Deposition Modeling (FDM) printed electrodes constructed using soft, biocompatible thermopolyurethane (TPU)-based carbon and graphite-doped materials, ensuring long-term wearability and minimal skin irritation, overcoming the drawbacks of gel-based electrodes. In embodiments described herein, the electrode of the present invention is integrated with wearable biosymbiotic systems and textile-integrated systems. To solve geometrical challenges when introducing multi-lead electrophysiology, the present disclosure also provides textile integration of biosymbiotic electronics and electrodes to enable dispersed connectivity of the torso, etc. The systems of the present disclosure include circuitry for at-distance wireless recharging to enable indefinite operation required for diagnostic targets. The systems of the present disclosure enables ECG, Electrical Impedance Pneumography (EIP), and Electrical Impedance Myography (EIM) (collectively referred to herein as “biomeasurements”) to directly evaluate physiological changes over chronic timescales relevant for comprehensive electrophysiological assessment without impact on daily activities.

### Biosymbiotic Systems Overview

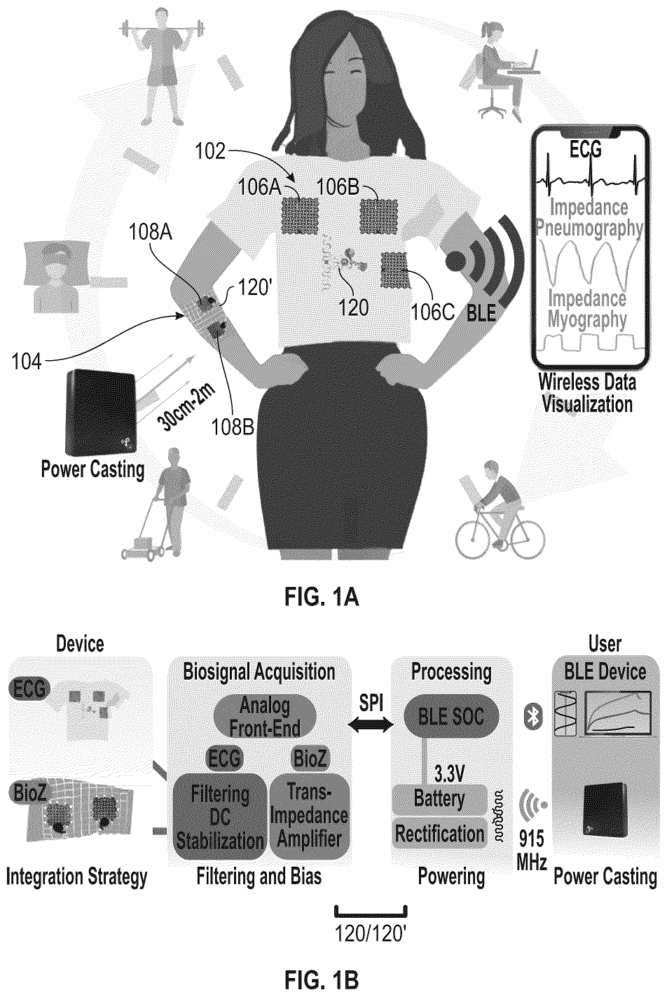

FIGS. 1A-1B illustrate an overview and operational characteristics of systems provided herein. In general, the electronics are characterized by conformal skin-interfacing facilitated by elastomeric materials in a meshed structure with system level elasticity matching the epidermis, wireless recharging at distance capabilities, and continuous data streaming. Enhancing this platform to record electrophysiological biosignals requires a technological solution for electrodes that are both soft, 3D printable and enable epidermal contact without adhesives or gels. The Biosymbiotic systems of the present disclosure utilize dry electrodes using fusion deposition modeled (FDM) 3D-printing with TPU-based, carbon-doped elastomer enabling integration with Biosymbiotic devices. 3D printing also enables precise control over geometry, enabling footprint and contact area optimization to best suit end use-case. The electrical and mechanical stability of this material in biosymbiotic electrodes enables use over indefinite timescales facilitating chronic, round-the-clock measurement of biosignals (as illustrated by the “wireless data visualization” plots of FIG. 1A). By way of example, in some embodiments the biosymbiotic systems of the present disclosure may be integrated with a textile (as generally illustrated in the shirt-integrated biosymbiotic devices 102 in FIG. 1A). In other embodiments and/or the teachings of the present disclosure may be accomplished using a wearable elastomeric biosymbiotic systems, as generally illustrated by the forearm wearable device 104 of FIG. 1A. Such wearable embodiments are shown and described in detail, for example, in U.S. patent application Ser. No. 17/791,523, filed Jul. 7, 2022, and U.S. patent application Ser. No. 18/281,527, filed Sep. 11, 2023, each of which are hereby incorporated by reference in their entirety.

The textile integrated biosymbiotic device, shown generally at 102, includes a plurality of integrated electrodes 106A, 106B, 106C placed on the front of the shirt and having a spacing useful for ECG and EIP measurements. The spacing between electrodes 106A, 106B, 106C is selected based on, for example, patient size and to optimize signal acquisition associated with ECP and/or EIG measurements. The wearable biosymbiotic device 104 includes electrodes 108A and 108B spaced apart and configured for impedance myography measurements. The details of the electrodes and the corresponding circuit components are described in greater detail below. In any of the implementations described herein, the teachings of the present disclosure enables long-term acquisition of electrophysiological signal, with textile integration useful in Electrocardiogram (ECG) and Electrical Impedance Pneumography (EIP) monitoring and integration with the biosymbiotic platform useful in performing Electrical Impedance Myography (EIM).

### Flexible Circuit Development

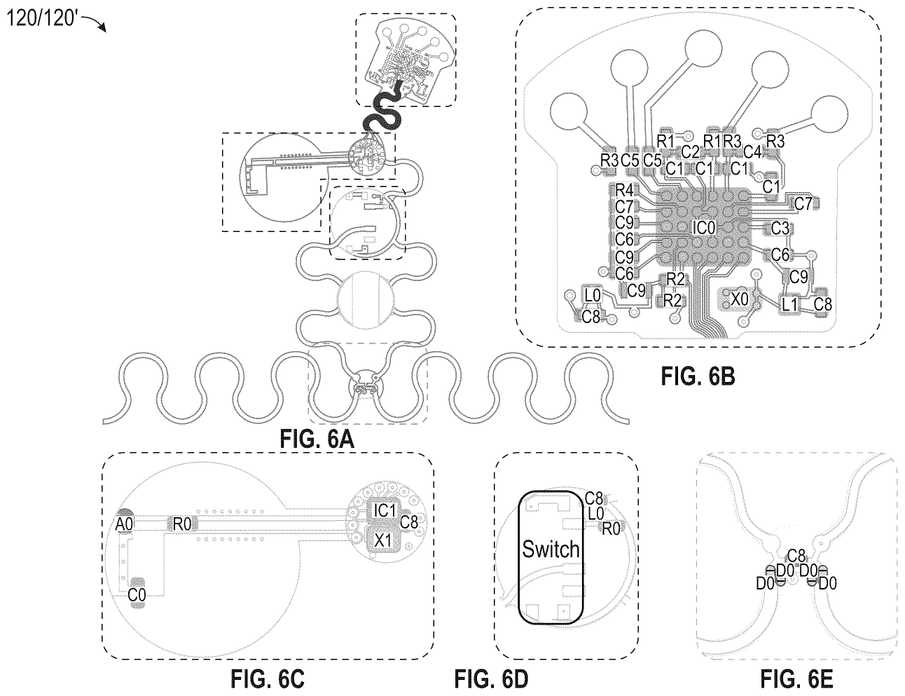

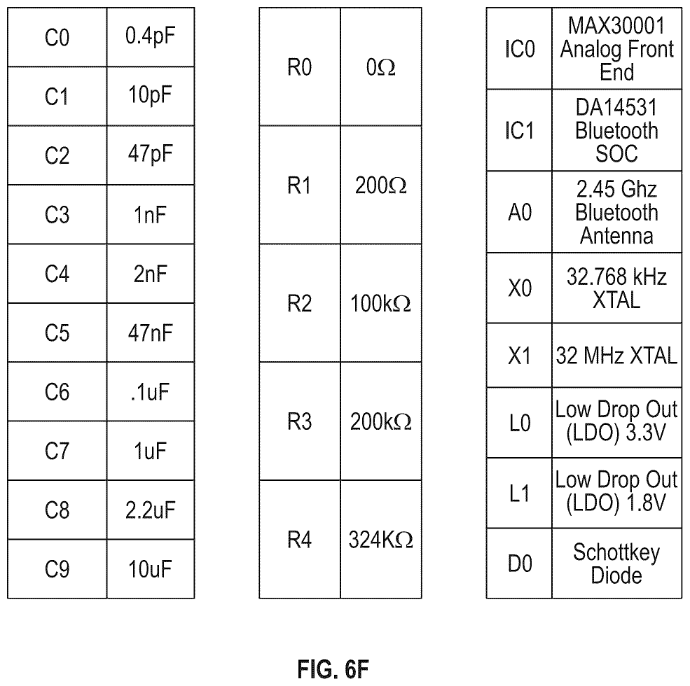

To support around-the-clock electrophysiology, miniaturized electronics with high energy efficiency may be used, as illustrated by the circuit block diagram of FIG. 1B. The circuitry is shown generally at 120 for the textile integrated embodiments of the present disclosure and 120′ for the wearable biosymbiotic embodiments of the present disclosure. Electrodes 106A, 106B, 106C, 108A and 108B are integrated through analog filtering which removes noise incurred from sources outside the physiological bandwidth, detailed in the Methods section below. An electrophysiology integratedCircuit (IC) featuring single-lead ECG and bioimpedance capabilities is used with the biosymbiotic electronic platform (see Methods sections, below) to continuously read out electrophysiological signals facilitated via Bluetooth low-energy (BLE) system on a chip (SoC). Data is compiled through smartphone, Raspberry Pi, or laptop computer. A commercially available power casting system that transmits power at 915 MHz is used to recharge the ultrasmall battery to provide a net power influx greater than energy usage throughout the day to enable uninterrupted operation without user interaction. Detailed circuitry 120/120′ and filtering protocols are described in the Methods section and illustrated in FIGS. 6A-6F.

### Biosymbiotic Wearable and Textile Electrode Fabrication

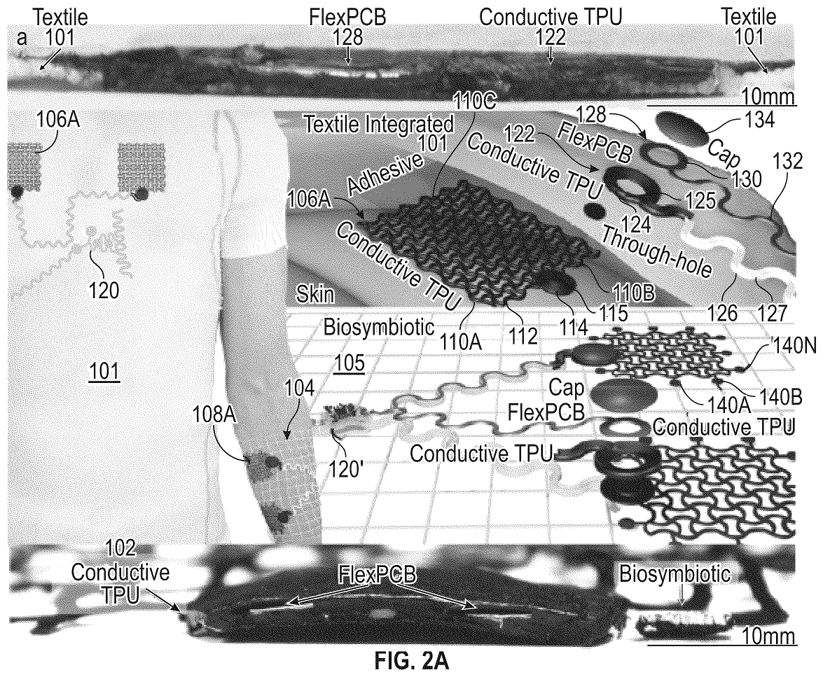

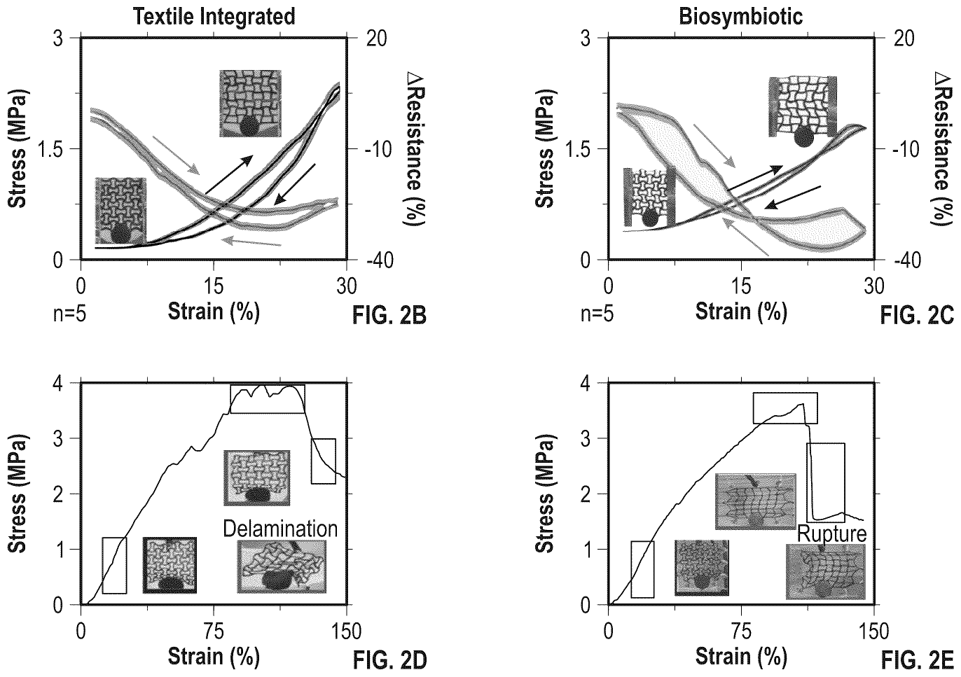

Key features for the dry electrodes of the present disclosure to perform well during daily activities is continuous epidermal contact which requires elasticity that matches the epidermis. This can be accomplished through both elastic materials and geometry. For biosymbiotic wearable and textile electrodes of the present disclosure, the devices may implement serpentine structures with functional units ranging from pseudo-ellipsoid to gyroidal in shape, as generally illustrated in FIG. 2A. As electrodes stretch, the serpentine geometry is chosen to minimize out-of-plane deformation and rely on the TPU elastomer to accommodate strain concentration. Combined, this enables a soft electrode that facilitates conformal contact with a high fill factor that supports transepidermal water loss for stable electrical contact. Textile integration, used for ECG and EIP biosignal collection follows this design strategy. Low out-of-plane deformation due to the intrinsic elastomeric electrode material is only limited in its stretchability by the integrity of the textile. Contact with the carbon doped TPU filament is challenging because mechanical mismatch and conformality under load are hard to maintain and conductive glues that are soft are not available.

FDM printing typically offers the opportunity to print freestanding, however, when combined with a porous substrate such as textile a tight integration of thermoplastic elastomer and the textile can be created (FIG. 2A). This technique enables large-footprint electrodes with interconnects that unify in a hub for electronics that can cover the entire torso. A challenge that arises with this technique is the necessity of a passthrough that enables signals to travel through the textile to mount outward-facing electronics. A thorough description of biosignal passthrough from skin to electrode to device is detailed in the Methods section. This method specifically developed for this device class includes heat-bonding the biosymbiotic conductive electrode to FlexPCB copper with structures to maximize surface area, and a cap and channel made of the same 3D printed material through a small hole created in the textile outlined schematically in Supplemental FIG. 2B with detailed description in the methods section. A cross-section of this integration is shown in the top panel of FIG. 2A. This provides signal passthrough in a mechanically stable, low-profile form-factor. Printing directly into textile represents only one possible integration strategy. An evaluation of the adhesive strength of eight proposed textile integration strategies is illustrated in FIG. 8.

FIG. 2A illustrates details of the electrode integration and connectivity for the textile-integrated devices (left-most panel, upper panel and upper middle panel of FIG. 2A) and the biosymbiotic wearable devices (left-most panel, lower panel and lower middle panel of FIG. 2A) according to various embodiments of the present disclosure.

Referring to the textile integrated devices, the upper middle panel of FIG. 2A illustrates a perspective view of the electrode 106A. The electrode 106A includes an electrically conductive mesh structure (described in greater detail herein) formed of a plurality of pseudo-ellipsoid to gyroidal filaments, e.g., 110A, 110B, 110C that cross to form roughly rectangular openings, e.g., 112 between each of the pseudo-ellipsoid to gyroidal structures. The overall size of the electrode 106A and the overall “tightness” of the mesh structure (defined as, for example, the number of openings per unit area, mesh size, etc.) may be selected based on the particular biomeasurement of interest, as described in greater detail below. The electrode 106A also includes a coupling pad 114 to couple the electrode 106A to electronic connectivity elements and to electrical components (shown generally at 120 and described below) and to secure the electrode 106A to a textile. The coupling pad 114 includes a crown portion 115 (approximately centered on the pad 114) configured to mate with electronic conductivity elements, described below. The electrode 106A is disposed on the underside of the textile 101 to ensure the electrode 106A remains in contact with skin, or the textile 101 has a patch removed approximately equal in size to the electrode 106A.

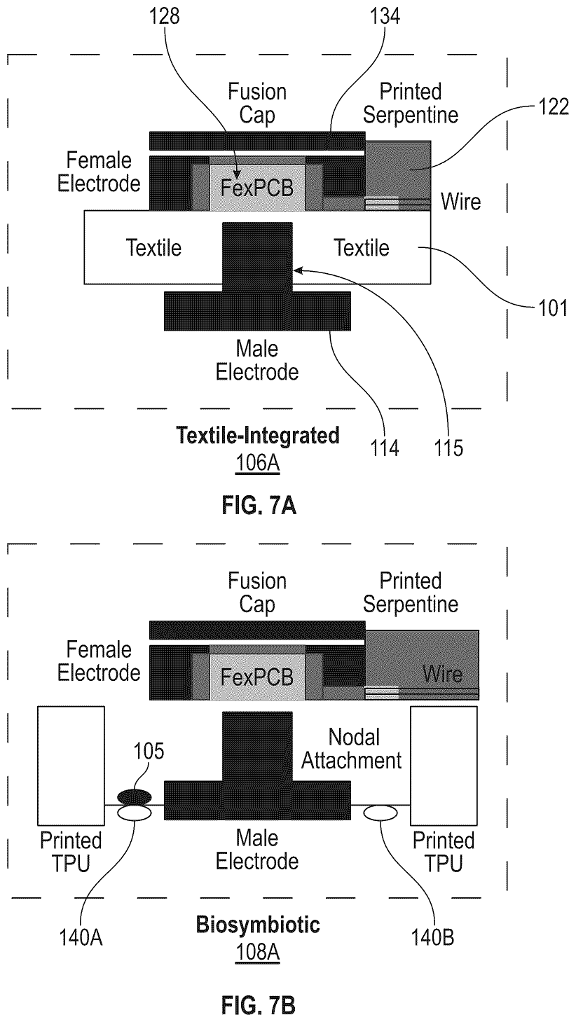

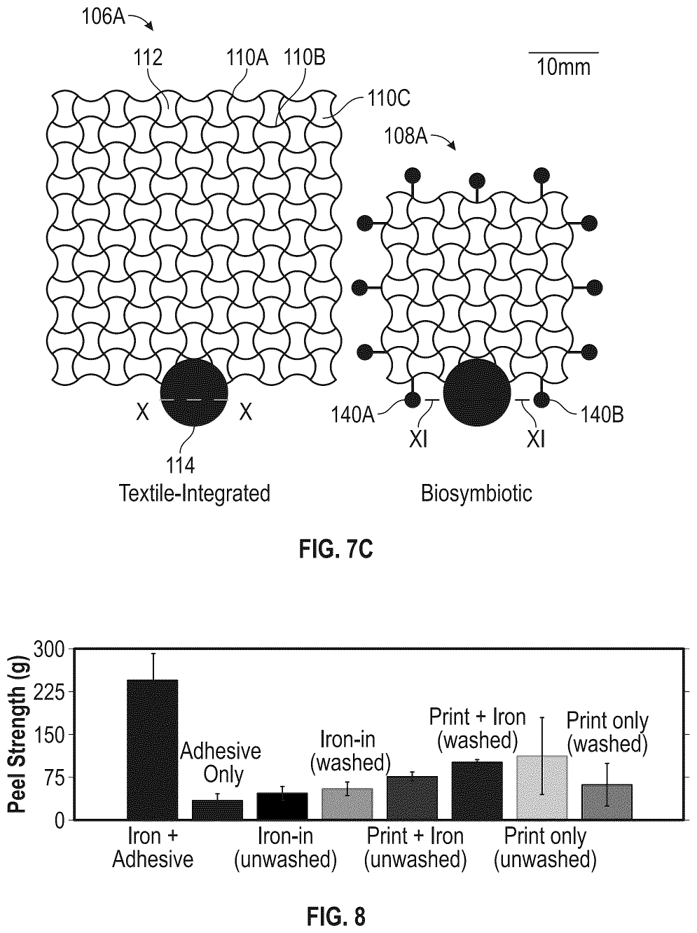

Electrical connectivity elements (as best shown in the upper middle panel of FIG. 2A) include a conductive TPU member 122 having a ring-shaped coupling member 124 defining an opening 125 and serpentine channel member 126 extending from the coupling member 124. The serpentine channel member 126 defines a channel 127 running along the length of the serpentine channel member 126. Electrical conductivity elements also include a flexible metallic member 128 (e.g., PCB material, copper wire, etc.) having a ring-shaped member 130 generally dimensioned to be received within the ring shaped coupling member 124 and a serpentine metallic member 132 extending from the ring shaped member. The serpentine metallic member 124 is generally dimensioned to be received in channel 127, and encapsulated as described herein. A cap member 134 is configured to be received into the annular opening of ring members 124 and 130 and to engage the crown portion 115 in a snap-fit arrangement via a through hole 103 in the textile 101. Thus, the conductive TPU member 122, flexible metallic member 128 and cap member 134 are disposed on the outside of the textile (or, in the case of a multi-layer textile, these components may be disposed between one or more layers). The electrical connectivity elements electrically couple the electronic components 120 (described herein) to the electrode 106A. The upper panel of FIG. 2A illustrates a cross-sectional view of the placement of the conductive TPU member 122 and flexible metallic member 128 through the textile fabric 101. Referring to FIGS. 7A and 7C, electrode 106A (FIG. 7C) is illustrated and FIG. 7A illustrates a cross-sectional view of the electrode 106A taken along line X-X (FIG. 7C, left panel) and showing the electrical connectivity elements described above. The electrodes 106B and 106C of this embodiment may be connected to the circuit components 120 in a similar manner as described above.

Referring again to FIG. 2A, the biosymbiotic wearable device 102 is illustrated (left panel) and the lower middle panel illustrates the electrode 108A of the wearable biosymbiotic embodiments of the present disclosure. The electrode 108A is similar to electrode 106A (described above), except electrode 108A also include bonding pads 140A, 140B, . . . , 140N formed around the periphery of the electrode 108A, and in particular, bonding pads 140A, 140B, . . . , 140N are respectively formed at or near the ends of the serpentine structures of the electrode 108A. The bonding pads 140A, 140B, . . . , 140N are configured to fuse (e.g., melt) onto the flexible mesh structure 105 of the wearable device 102. In some embodiments, the mesh structure 105 is at least partially removed under the electrode 108A to ensure skin contact with the electrode 108A. The electrical connectivity elements for the wearable device 102 are similar to the electrical conductivity elements 114, 122, 128, 134, except that, in this embodiment, the electrical conductivity elements are not coupled together through fabric. Referring to FIGS. 7B and 7C, electrode 108A (FIG. 7C) is illustrated and FIG. 7B illustrates a cross-sectional view of the electrode 108A taken along line XI-XI (FIG. 7C, right panel) and showing the electrical connectivity elements described above, and the bonding pads 140A, 140B, . . . , 140N coupled to the mesh 105. The electrode 108B of this embodiment may be connected to the circuit components 120′ in a similar manner as described above.

### Electronics Integration

Electrical impedance myography for reliable muscle bioimpedance requires a biosymbiotic attachment to accommodate limb mounting location that enables free garment choice. We use digital design processes described in previous work to generate skin-conformal device interaction. This includes designs for the energy harvesting antennas and associated circuits that handle data acquisition from the electrophysiology front end and wireless communication via BLE as outlined schematically in FIG. 1B. These electronic components 120/120′ are located on flexible PCB that are encapsulated into the TPU biosymbiotic structure. Detailed information on the electronics used and associated fabrication schemes is found in the Methods section below.

### Mechanical and Resistive Response to Physiological Loading Regimes

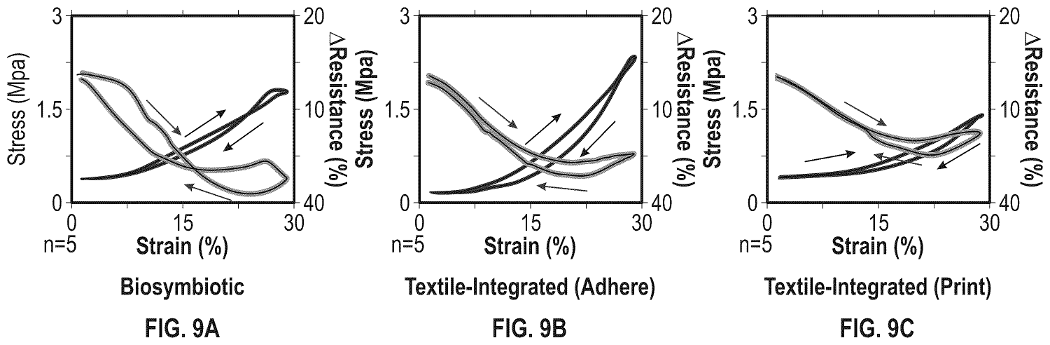

FIG. 2B illustrates mechanical and resistive reactions to applied strain of the electrode 106A in a textile-integrated platform. Results represent five cyclic loading instances to 30% strain, where shaded areas represent standard deviation (SD) from the mean in repeated loading for both stress and resistance readings. Stress readings drop on the relaxation cycle, which is an intrinsic property of the TPU-based elastomer. The hysteresis curve shows a return to baseline for stress output, and a negligible (1.09%) change in electrode resistance after a complete load cycle. As strain increases, inherent resistance drops, most dramatically a 31.4% drop at 22.5% strain. This behavior is observed in other TPU conductive material blends in literature, and behaves similarly to results obtained for different textile-integration strategies as shown in FIGS. 9b and 9c. Deforming a layered carbon structure generates a piezoresistivity effect caused by geometrical changes creating greater contact between the randomly distributed carbon molecules due to the Poisson effect. This explains the disparity between resistive response for the textile-integrated platform and biosymbiotic mesh-integrated platform. As shown in FIG. 2C, stress and resistive response to loading show a similar trend with a more pronounced resistive hysteresis response, peaking at 36.3% decrease in intrinsic resistance. Mechanistically, this points to an enhanced localized strain in the freestanding serpentine, improving conductivity, in-line with similar results to carbon nanotube stretching curves. The small change in electrode resistance that is always lower than skin interface resistance highlights the robustness of this approach with no observable difference in performance regardless of strain state.

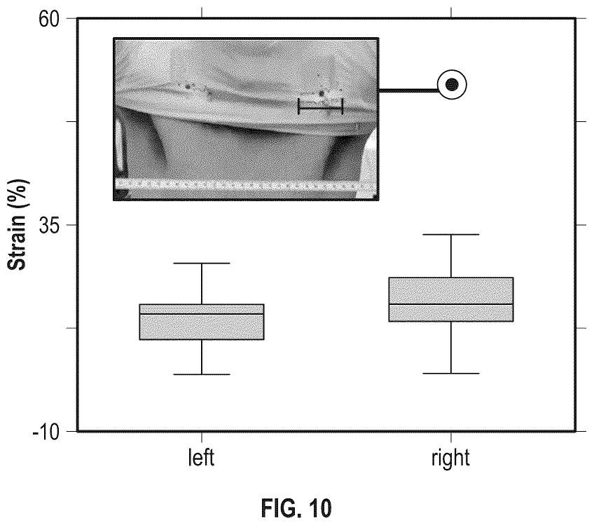

Ultimate failure strain is shown in FIG. 2D highlights the textile-integrated platform as a composite failing at 3.94 MPa, with the failure mode of delamination (inset, FIG. 2D), occurring at over 100% strain. Repeated donning and doffing of a textile-integrated device yielded a maximal strain value of 48.8% at the extreme, (FIG. 10, inset), with typical loading profiles closer to 20%, matching expected values of 30% or less. Electrodes integrated for the wearable biosymbiotic platform fail at the junction between TPU and electrode filament. Shown in FIG. 2E, electrodes remain elastic until the incurred strain reaches 97%. Both integration strategies perform similarly mechanically and maintain structural integrity well beyond expected physiological loading scenarios.

### Electrode Geometry Optimization

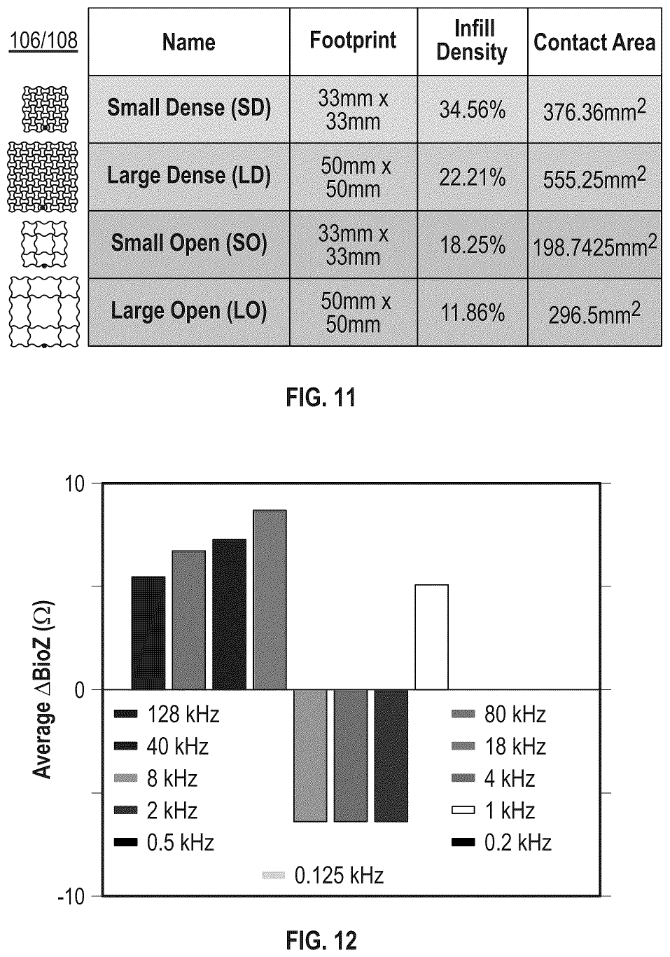

FDM printing offers near-limitless control over electrode geometry. For simplicity, four biosymbiotic electrode geometries are evaluated against the gold standard gel electrode. As contact area and its influence on contact impedance determine the effectiveness of a dry-electrode interface, the teachings herein evaluate two electrode footprints (termed Large and Small), and two electrode relative infill densities (Dense and Open). Photographs and geometric characteristics of each electrode type as well as their acronyms are found in the table of FIG. 11.

### ECG Characterization

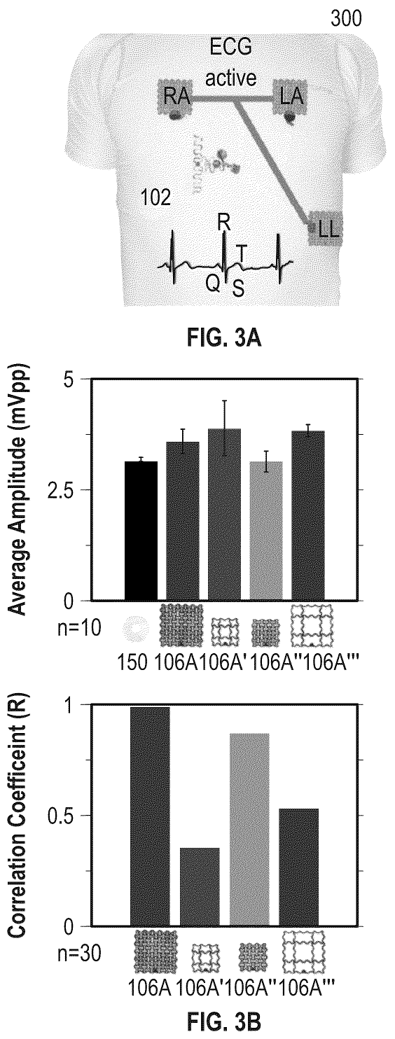

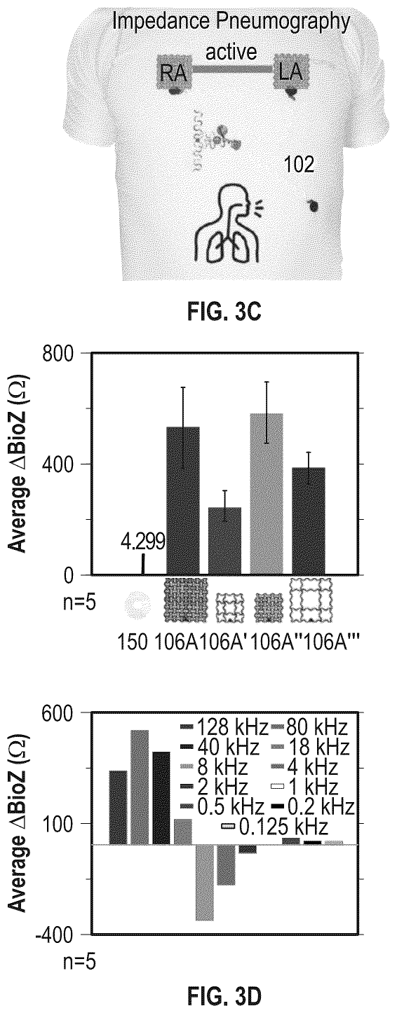

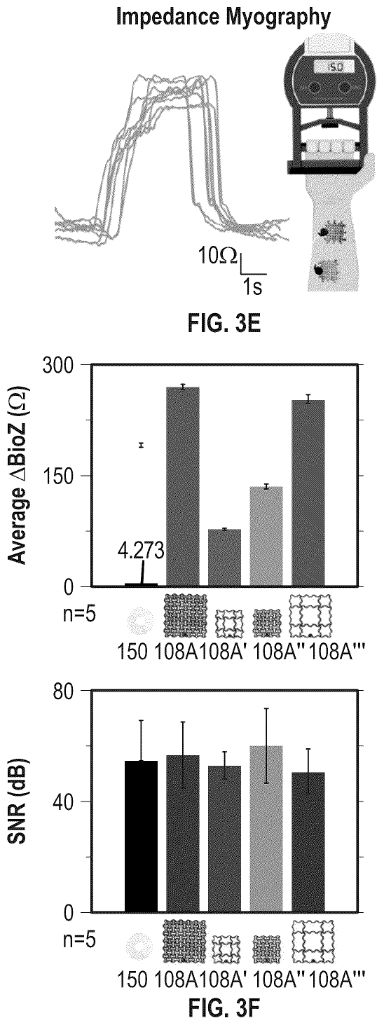

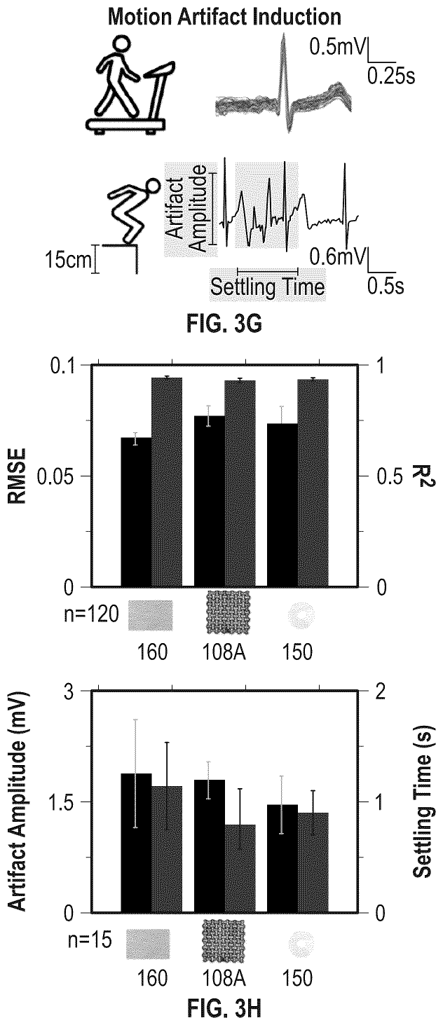

Electrodes printed into a workout shirt with compression fit in the locations highlighted in in FIG. 3A illustrates ECG performance. Peak-to-peak (p-p) amplitudes, given by summation of R and S peak absolute value on a resting subject is used for evaluation. FIG. 3B (left panel), shows average p-p value of each electrode type 106A, 106A′, 106A″ and 106A′″ from 10 heartbeats. All printed electrode types performed equivalent or better than gel electrodes 150, shown in FIG. 2B. From these results, the small open electrode (106A′) produces the highest signal amplitude. Another metric for performance relevant for clinical use of ECG for diagnosis of arrhythmia, ischemia, and conduction disorders depends on feature identification. As such, correlating biosymbiotic electrode captured signal to signal from gold standard is the determining metric for top-performing electrode geometry. Correlations of simultaneous measurement of biosymbiotic and gel electrode signals are shown in FIG. 3B (right panel). Overlaid waveforms show excellent feature preservation when comparing dense electrode configurations, with correlation values of 0.9841 (98.41%) for the larger footprint 106A, and 0.8719 (87.19%) for the smaller footprint 106A″. These results suggest that a larger electrode contact area and higher electrode densities preserve key features of the ECG waveform better crucial to its use as a diagnostic tool.

### Electrical Impedance Pneumography Characterization

For EIP, electrodes have similar low-frequency requirements compared to ECG electrodes with the added requirement of good current injection capabilities. In FIG. 3C, two electrodes equidistant from the sternum acquire EIM signals in our textile-integrated platform. FIG. 3D (left panel) shows average amplitude change over 5 complete tidal breath cycles. The electrodes 106A, 106A′, 106A″ and 106A′″ yield significantly higher tidal impedance amplitudes than that of the gold standard electrode 150, indicating a stronger response to volumetric changes across the thorax. These results suggest that electrode density is a determining factor in enhanced signal strength, with the most dense (SD) geometry showing highest signal amplitude.

This modality additionally facilitates optimization of current injection frequency for highest signal peak-peak amplitude. In the right panel of FIG. 3D, steady impedance readings from a stable maximal inspiration breath hold following a maximal expiration characterize optimal current injection frequency. For a fixed current injection amplitude of 32 μA, a drive frequency of 80 KHz yields an average impedance change of 516.902, 93.12 higher than the next most responsive frequency which is a consequence of tissue penetration coupled to the respective frequencies and the equivalent circuit of the biosymbiotic electrodes that feature larger capacitive components compared to traditional gel electrodes. The same experiment, shown in FIG. 12 yields maximal p-p impedance for a tidal breath of 8.64Ω occurring at 18 kHz instead of 80 kHz. At lower frequencies (500-125 Hz), tidal breath is indistinguishable from noise.

### Electrical Impedance Myography Characterization

Evaluating EIM performance relies on a series of repeated muscular loading, where a subject squeezes a hand dynamometer to 15 kg for five seconds. Biosymbiotic electrodes aligned with the brachialis muscle, are used to capture resulting impedance changes. A schematic of the loading regime, example waveform overlays, and a biosymbiotic mesh platform 104 is shown in FIG. 3E. Average signal amplitude measurements are defined as the difference between mean stable reading at baseline and mean stable reading during muscle contraction. Results show for electrodes 108A, 108A′, 108A″ and 108A′″, the electrode footprint size is the dominant factor in signal amplitude, with the two largest electrodes generating nearly double the resistance change compared to smaller, Dense (SD) configuration, shown in FIG. 3F (left).

The same test facilitates the acquisition of Signal-to-noise ratio (SNR). Mean signal strength corresponding to a 15 kg hand dynamometer hold is termed “signal,” where noise floor (noise) is defined as the average without hand loading. SNR is relatively similar across all electrode types with the highest reported signal-to-noise ratio measured with the small dense electrode with a value of 60.07 dB (FIG. 3F, right).

### Motion Artifact Characterization

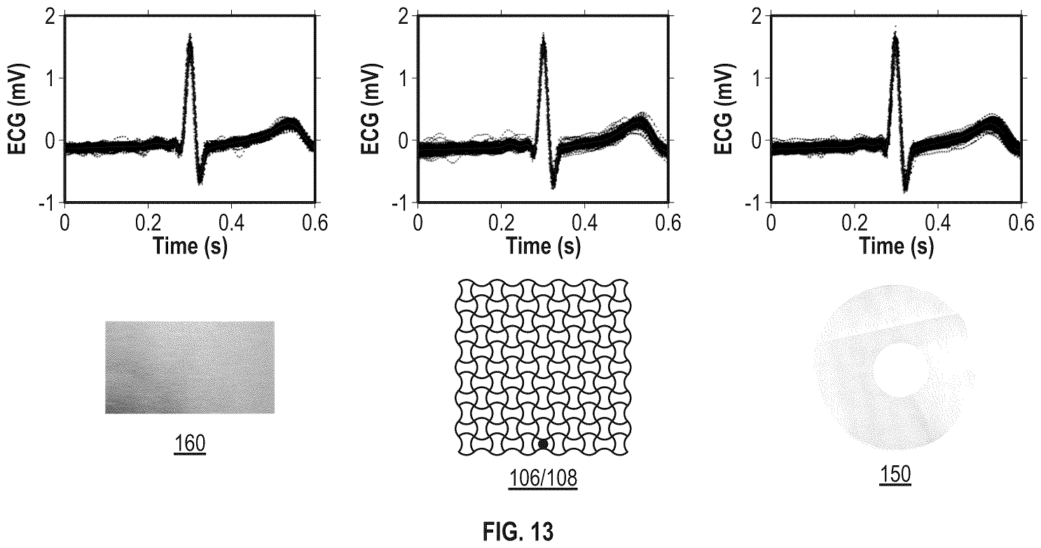

Current barriers to widespread adoption of dry electrodes in chronic electrophysiological monitoring relate to their proclivity for signal disturbances from motion. The usefulness of a biosymbiotic electrode system is inherently dependent on perturbations of signal when subjected to daily activity. FIG. 3G details schematically two instances of motion artifact induction to evaluate the biosymbiotic electrodes. Motion type 1 is represented by a treadmill walk, and motion type 2 is extreme motion artifacting instances, represented by a jump from a platform. Type 1 tests yield Root Mean Square Error (RMSE) for 120 overlaid heartbeats compared to smoothing spline fit to the aggregate signals. All three evaluated electrode types show excellent signal reliability with RMSE ranging from 0.067 in the gold standard dry electrode 160 geometry cut from stainless steel (SS), such as used in continuous heart rate monitors for sports application, to 0.073 for Ag/AgCl, to 0.077 for biosymbiotic electrodes. Signal correlation follows a similar trend, with R² values of 0.940, 0.932, and 0.927 for SS, Ag/AgCl, and biosymbiotic electrodes, respectively. These signal correlations represent a 1.4% difference from the highest to lowest performing electrode, all shown in the left panel of FIG. 3H. A visual comparison of the three overlaid waveforms and their line of best fit is shown in FIG. 13. All three electrode types show reliable signal quality and resistance to signal-disturbing motion artifacts in a controlled walk test.

Type 2 motion effect on signal quality is particularly relevant in biosymbiotic systems designed for 24/7 use. Fifteen jumps from a 15 cm tall box show each electrode's performance in absolute motion artifact amplitude, measured as the sum of maximal deviation from baseline in both positive and negative mV. In black of the right panel of FIG. 3H shows gel electrodes perform highest in resistance to high amplitude motion artifacts, with an average artifact amplitude change of 1.46 mV. The biosymbiotic electrode of the present disclosure performed 5.46% better and with higher consistency (SD 96.2% less) than SS electrodes in extreme motion, with an average artifact amplitude of 1.78 mV. This shows signal acquisition performance of higher quality than the commercial standard. Another metric that can be captured to evaluate the noise is signal reacquisition, termed settling time. In red, average time to settle for each electrode type shows biosymbiotic electrodes performing 11.9% better than Ag/AgCl electrodes and 36.4% better than SS electrodes with an average settle time of 0.7861 seconds. For chronic-use applications, an electrode with rapid signal reacquisition is ideal, as physiological movements that affect signal quality are minimized.

This suggests the biosymbiotic electrodes according to the teachings of the present disclosure perform on par or better than gold standard electrode types for motion-induced disturbances.

Chronic Stability of Biosymbiotic Electrodes with ECG Measurements

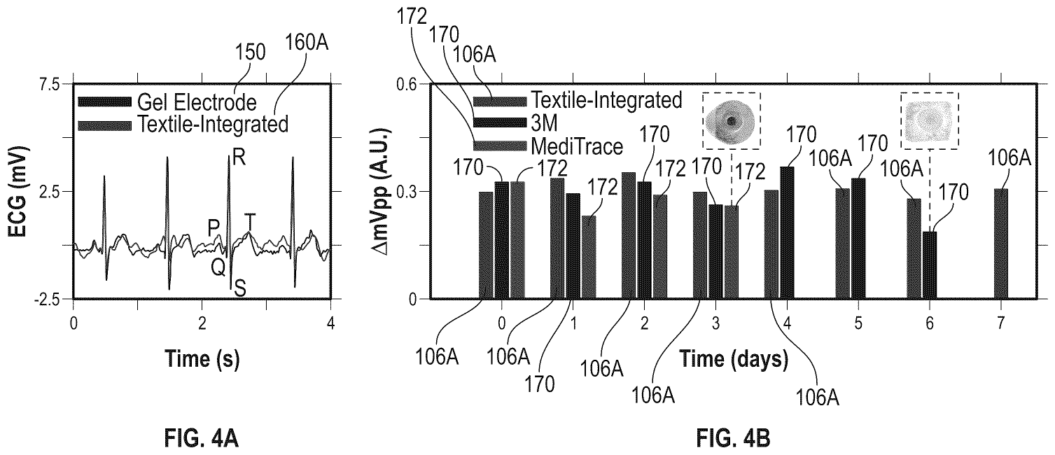

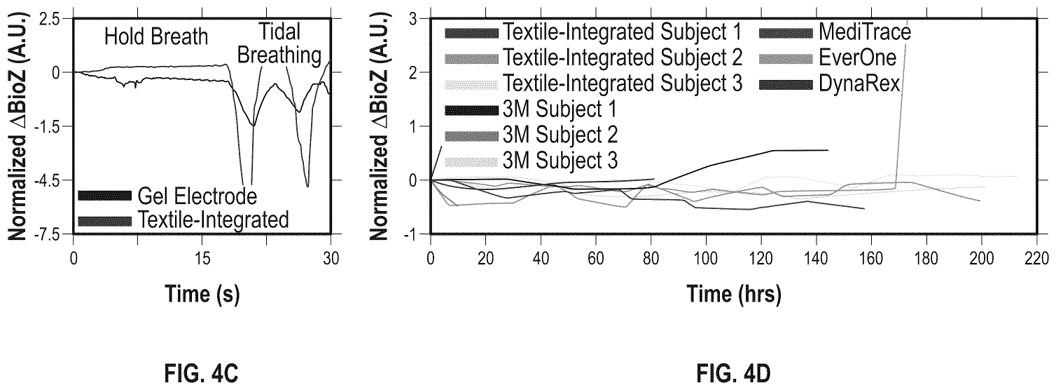

In a chronic biosignal monitoring system, signal integrity must be maintained throughout the measurement period, a known weakness for gel electrodes and adhesive-based systems. Experiments are performed that measure the biosymbiotic electronics of the present disclosure against gel gold standards. An example of signals at 144 hours of continuous wear is shown in FIG. 4A. Results from a continuous wear test with normalized peak amplitude trends is shown in FIG. 4B. Two high-performing gel electrode brands (3M 170, MediTrace 172) are asynchronously evaluated for ECG signal integrity until gel electrode failure, details in methods section. Each electrode type (108A, 170, 172) is normalized by the mean peak-peak amplitude of 60 s of continuous resting ECG. Electrode performance remains relatively constant until adhesive failure of the gel electrodes. Failure mode of gel electrodes (FIG. 4B, inset) shows a significant amount of dead skin and hair accumulation on the adhesive layer characteristic for regular epidermal turnover. The Biosymbiotic electrodes 108A of the present disclosure remain stable without degradation.

Chronic Stability of Biosymbiotic Electrodes with Impedance Pneumography

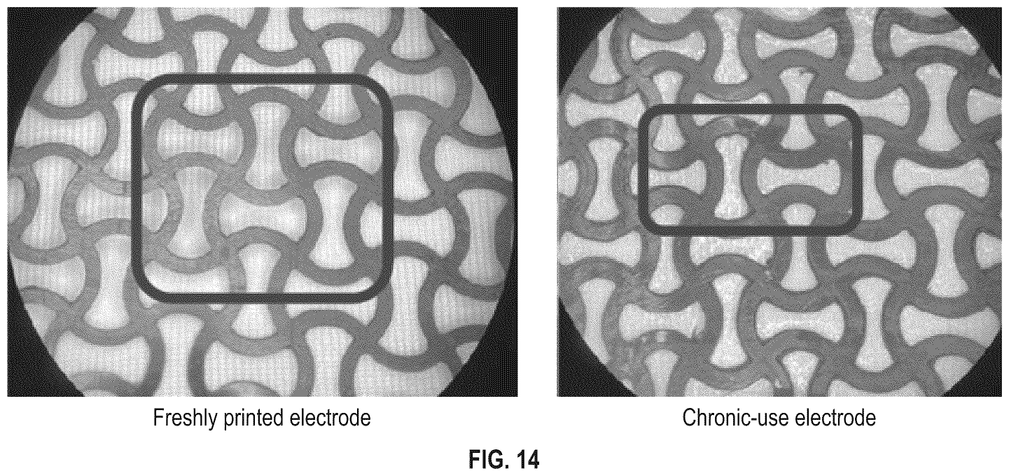

FIG. 4C shows a waveform overlap of normalized-by-mean skin-impedance values of biosymbiotic electrodes and gel electrodes. Chronic stability characteristics for the textile-integrated biosymbiotic electrode platform are plotted against a multitude of gel electrode brands (3M, MediTrace, EverOne, DynaRex) in FIG. 4D. Electrode failure of the budget gel electrodes is due to dehydration while high-performance gel electrodes last about the same time as the chronic ECG measurements. FIG. 4D shows continuous wear appears to improve performance, shown by a net 117.8% decrease in skin-electrode impedance and a net increase in electrode impedance for a high-performing gel electrode (3M) of 35.4%. This could be due to localized sweat deposition of electrolytes and cell matter on the biosymbiotic electrode surface facing the skin shown before and after extended wear in FIG. 14, and sustained fatigue loading of electrode in shirt causing electrode relaxation and better conformality to skin, suggested by the results in FIG. 2B. For ECG measurements, this small change in impedance is not visible as it does not affect signal quality. Essentially, FIG. 14 illustrates that a chronically-used electrode (left panel) shows very little wear compared to a new electrode (left panel), and any such wear has not been shown to degrade signal quality in any meaningful way.

### ECG Compliance to Gold Standard Measurements

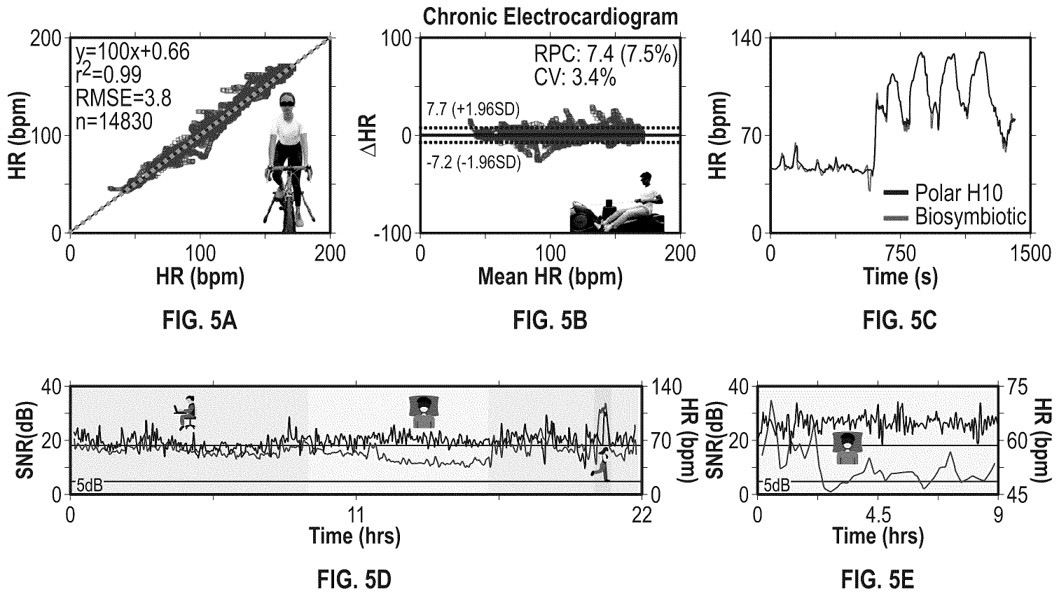

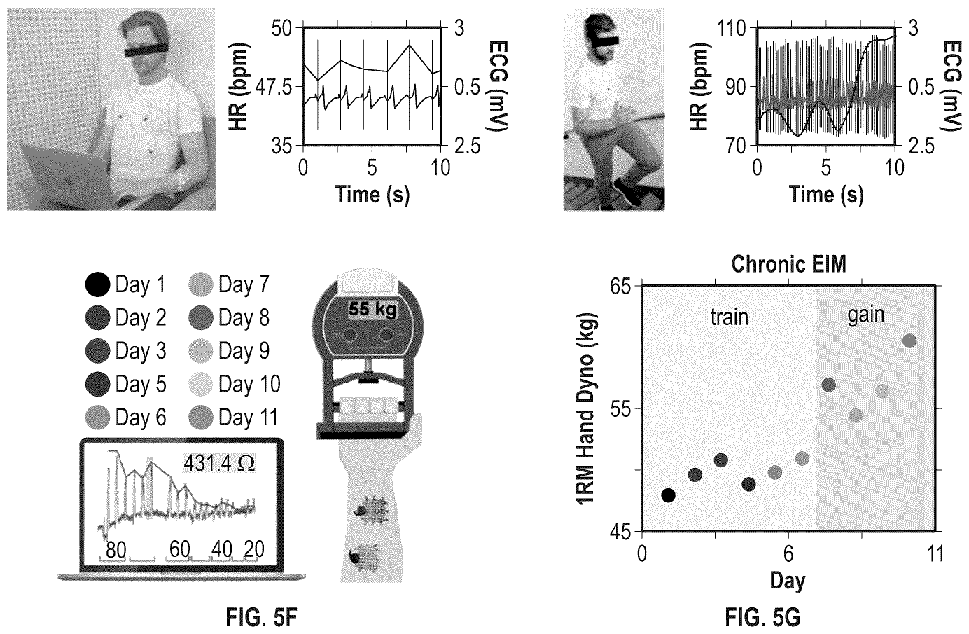

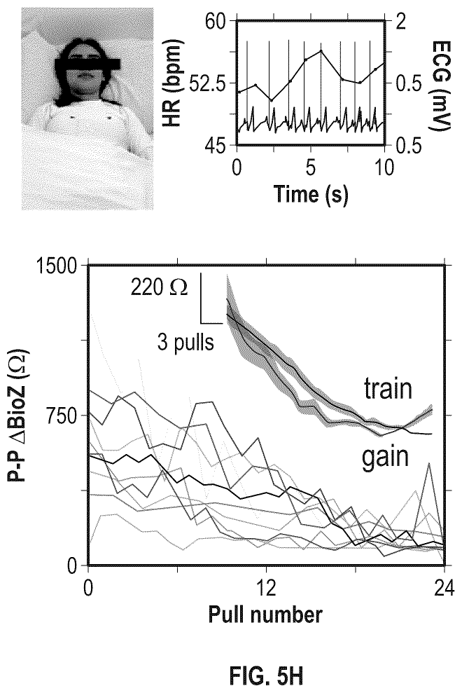

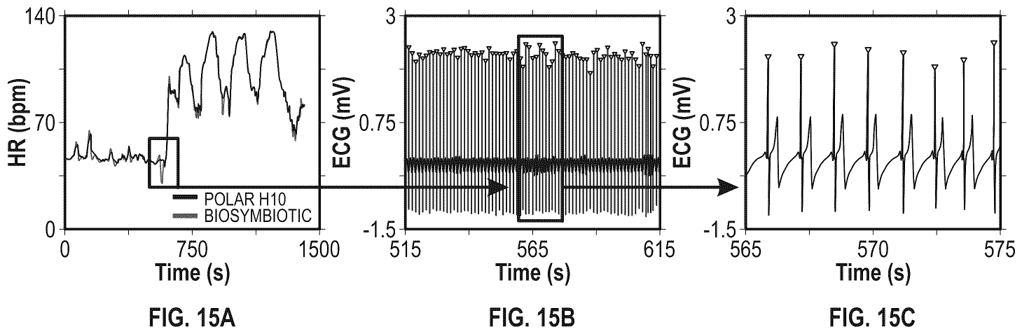

Demonstrating system efficacy in real-world use cases, the textile-integrated biosymbiotic device is evaluated by continuous ECG for feature extraction and comparison against a gold standard activity tracker (Polar H10). Heart rates extracted from activities such as stationary cycling, rowing, and running up and down stairs strongly correlate with heart rate data extracted from the gold standard. Data collected from two male subjects and two female subjects show excellent correlation to gold standard when relevant features were extracted in post-processing, evidenced in the Pearson correlation plot shown in FIG. 5A by the excellent coefficient of determination (R²=0.99). The Bland-Altman plot analysis presented in FIG. 5B illustrates excellent adherence to HR calculated by the gold standard, with a coefficient of variance of (3.4%), indicating consistent performance across the range of analyzed HRs. FIG. 5C shows an example of simultaneously tracked data from the gold standard and individual HR readings from the textile-integrated biosymbiotic device, qualitatively detailing both temporal and spatial resolution of the collected signal. FIGS. 15A-15C shows the graph in FIG. 5C with a callout where collected heart rates visually deviate from the gold standard and shows a zoomed-in view of the raw ECG data over 100 seconds starting 50 seconds before this instance, with a third callout of ECG signal acquisition during this period showing perfect signal acquisition and peak identification. This highlights enhanced performance by the textile-integrated system of the present disclosure over the gold standard capable of identifying spikes of heart rate and abnormal events currently missed by gold standard devices.

Chronic performance in daily activities are demonstrated with a continuous monitoring experiment. To simulate typical use cases, a data collection period of 22 hours, corresponding to one full day including work, activity, and sleep, show high continuous signal fidelity and heart rate calculations for a male subject. FIG. 5D shows a plot of signal quality measured by signal-to-noise ratio with continuous measurement fidelity at or above the required threshold of 18 dB for complete clinical grate ECG waveform analysis and far above the necessary strength of 5 db for accurate QRS complex detection. The averaged heart rate readings over the same timescale illustrate perturbations to heart rate as the subject performs daily activity such as desk work, shown in the left inset of FIG. 5D, with recognizable trends such as activity, shown in the right inset of FIG. 5D. Observable elevation in HR during daily activity, drop during sleep, and pronounced increase at wake match well-characterized traits of healthy physiological circadian rhythm. A second use-case analysis of a sleep cycle for a female subject shows reliable signal quality over nine continuous hours with SNR averaging 26.3 dB, with only one average reading dropping below the critical threshold of 18 db (15.52 dB), still allowing accurate QRS complex detection. Pronounced HR drop consistent with variations in stable reading while sleeping further illustrates the textile-integrated bioelectronic reliability with known physiological phenomena. Given biosymbiotic system performance and known complaints of comfort for patients utilizing a Holter monitor, these results offer much promise for improving patient compliance and natural sleep patterns while recording continuous ECG.

### Chronic Recording of EIM

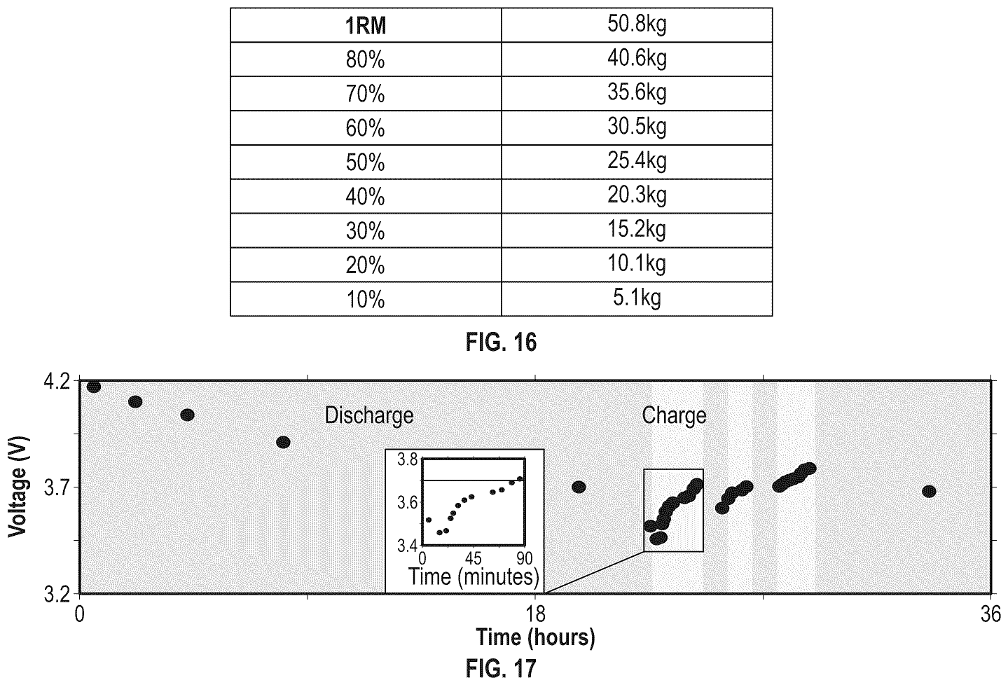

Wearable systems evaluating EIM are largely unexplored, with only two published works in the past four years exploring this modality. Both systems fail to report chronic measurement capabilities and utilize gel electrodes. The teachings of the present disclosure demonstrate the ability to record muscle signals from the forearm of a subject with high spatial and temporal resolution. To demonstrate these capabilities in chronic, indefinite EIM recording, a subject wears a biosymbiotic electrode system continuously for eleven days and follows a strength training protocol performed with a hand dynamometer. FIG. 5F outlines example data correlating peak-peak resistance readings with increasing amplitudes of hand dynamometer pulls and electrode placement over the brachialis (described in Methods section, below). FIG. 5G shows, in kilograms, the one rep max (1 RM) gripping instance per each day of the test, performed upon wake in the morning. After six days of training, a clear gain in strength is shown, allowing to group data from the first six days as “training data,” shown as a black overlay and the last four days as “gains data,” with red overlay. More details on the strength training protocol may be found in the Methods section with an example protocol in the table of FIG. 16. Powering of the device was accomplished with far field power transfer which is described in the aforementioned US patent applications. An example of battery discharge and recharge curve for a typical cycle is shown in FIG. 17.

While a direct linear trend of muscle strength gains and signal amplitude is within the noise margin, FIG. 5F shows clearly distinguishable sensor signal strengths associated with modulations in weight pulled on the hand dynamometer. This is the first demonstration of this technology, showing high-quality signal strength with uninterrupted epidermal interfacing over chronic timescales, demonstrating the lack of conscious interaction required. When aggregating sensor readings for prescribed hand dynamometer pulls, there is a trend of decreased peak-peak bioimpedance when comparing the mean aggregated “gain” data against mean aggregated “train” data (FIG. 5H, inset), suggesting the use of this method of chronic monitoring to quantify muscle state. There is limited data to suggest that increased handgrip strength leads to lower resistance in EIM measurements, a trend that repeats here. This change likely relies on enhanced motor unit recruitment and sarcomere overlap, as opposed to pure muscle hypertrophy. Investigation on the fidelity of such a system to characterize objectively muscle strength from bioimpedance may be accomplished with the teachings of the present disclosure.

Current challenges with dry electrodes, battery bulk, and capability for uninterrupted continuous data acquisition and need for user interaction with devices comprise key technological barriers to chronic acquisition of electrophysiological biosignals needed for diagnostics. To address these challenges, the textile-integrated and biosymbiotic electrodes according to the teachings herein may be integrated with wireless continuously powered low profile soft biosymbiotic electronics for electrophysiological diagnostics. The system is enabled by FDM printing of carbon doped TPU filaments that enable a scalable and personalized wearable solution. The teachings of the present disclosure demonstrate high-quality electrocardiogram signal acquisition in both male and female subjects, addressing a current gap in literature for system applicability to a diverse population. Textile-integrated device performance in continuous daily use and varied activity experiments show robust biointerfacing and clinical-grade data streams without user input or interaction.

Additionally, teachings of the present disclosure demonstrate the fidelity of the system with chronic muscle bioimpedance characterization highlighting fidelity which may be used in future for defining fatigue state of an individual and possibly for predictions of maximal muscle output in real time. The technology also may enables real-time assessment of muscle hypertrophy, atrophy, and/or disease states.

### Materials and Methods:

### Flexible Circuit Fabrication

Flexible circuits designed with AutoCAD 2021 were laser-cut (LPKF, Protolaser U4) from FlexPCB panels (PCBway, Constituent layers: 12.5 μm Polyimide, 15 μm Adhesive, 12 μm Copper, 25 μm Polyimide, 12 μm Copper, 15 μm Adhesive, 12.5 μm Polyimide). After laser-cutting, PCBs were cleaned by sonication (Vevor, commercial ultrasonic cleaner 2 L) for 2 minutes in flux (Superior Flux and Manufacturing Company, Superior #71), followed by rinsing with deionized water. Commercially available components were placed by hand and reflowed with low-temperature solder-paste.

### Circuit Design, Fabrication

A serpentine shaped dipole antenna with resonance at 915 MHZ (described in previous work) with full bridge rectifier was built using commercially available components placed by hand and reflowed using low-temperature solder paste. The full-bridge rectifier used low-forward voltage Schottky diodes (Skyworks, SMS7630-061), a smoothing capacitor, and a 3.3V Zener Diode (Comchip Technology, CZRZ3V3B-HF) to rectify signal and provide overvoltage protection. A node composed of exposed copper strips allowed direct connection to a 3.7V, 100 mAh battery (product), which was charged using harvested power. A single throw single pull switch (product name) controlled device power. A 3.3V Low Drop-out (LDO) provided stable power and overvoltage protection to the microcontroller. A Bluetooth Low Energy (BLE)-capable microcontroller System on a Chip (SoC) (DA14531) was programmed by soldering flexible wires (Calmont) onto General Purpose Input/Output (GPIO) pins corresponding to SW_CLK and SW_DATA and using Dialog's SmartSnippet Studio program. BLE SoC interacted via Serial Peripheral Interface (SPI) communication with an ECG and BioZ analog Front-End IC (MAXIM, MAX30001). The ECG and BioZ Analog Front-End features two low-pass filtering circuits on the ECG signal lines at 79.58 kHz (200 kΩ, 10 pF), and a low-pass filtering circuit at 0.0796 Hz (200 kΩ, 10 μF), providing a near-DC consistent biasing signal. Signal is passed to the on-board differential amplifier through a 2 nF parallel capacitor, equalizing noise across both ECG electrodes. The BioZ signal line low pass filtering circuits at 79.6 MHz (20052, 10 pF), with common-mode noise rejection at 16.9 MHz. Data were communicated via BLE protocol at 2.45 GHz using an external chip antenna (YA-GEO, ANT1608LL14R2400A).

### Textile Integration of Electronics

The 3D printed serpentine electrodes interact with downstream electronics via a conductive Pyralux double-sided copper clad laminate (AG185010RY; constituent layers, 18 μm copper, 50 μm polyimide, and 18 μm copper) FlexPCB laser-cut serpentine element with a 4 mm² circular opening press-fit into a raised “male” circular pillar at the electrode x-midline and y-minima. This serpentine element is paired with a “cap and channel,” represented by a conductive 3D printed element that is a negative of the FlexPCB, and a printed cap that fits atop the circular pillar, also a press-fit. The electrode is assembled such that the circular pillar of the electrode body intersects with the channel component, FlexPCB is carefully inserted, and the cap is placed on top, creating a multi-layered structure of preprocessed height 1.2 cm. Once desired FlexPCB orientation is obtained, the four layers are heat-bonded into a single cohesive unit via a household clothing iron set to the highest temperature. A layer of parchment paper is laid on top of the assembled electrode and the iron is pressed down uniformly.

### Biosymbiotic Mesh Integration of Electronics

2D mesh drawings were exported from AutoCAD and imported a 3D modeling software (Autodesk, Fusion 360) for extrusion. These models were exported as Stereolithography (STL) files and imported into a 3D slicing software (Prusa3D, PrusaSlicer) which converts the model into machine code usable by a 3D printer. A fusion depositing modeling printer (Creality, CR-10S) was retrofitted to use an all-metal direct drive extruder (Creality, Sprite) with automatic bed-leveling unit (Antclabs, BLTouch). TPU (NinjaTek, NinjaFlex) was printed at 45 mm/s at an extruder temperature of 225C and bed temperature of 45 C. After printing, chronic end-use devices saw segmented sections joined by melting circular nodes of 5 mm diameter together. Short-term use-case biosymbiotic meshes were outfitted with clinging adhesive (Velcro) for rapid donning and doffing of device.

Electronics (Circuitry) Integration with Biosymbiotic Platforms

Relevant electronics as described in FIGS. 1A-1B integrate with the textile platform by FDM printing nonconductive thermoplastic polyurethane (TPU) (NinjaTek, NinjaFlex) of shore hardness 85A in a 0.45 mm thick island with 0.2 mm walls directly into the textile platform patterned to fit device footprint. To connect electrodes with the active electronics, serpentine channels also comprised of a 0.45 mm base and 3 mm tall are FDM printed directly onto a glass bed and adhered to the shirt via fabric adhesive (3M) which is subsequently ironed on the reverse side of the shirt. Ultraflexible wire (Calmont, 36 gauge) is woven into the TPU serpentines, then both device and wires are encapsulated with UV curable flexible resin (Superfast Superflex) of shore hardness 80A.

### Mechanical and Resistive Hysteresis Experiment

Mechanical Characterization of electrode integration strategies was performed with a custom 3D printed stretching stage. Tests were performed by securing the integrated substrate in a 3D printed mold (PLA) with an embossing (depth 1.2 mm) of the Small Dense electrode geometry, secured with a positive extruded section (height 0.75 mm) matching the electrode geometry. These two sections were bolted together to ensure equally distributed loading across the electrode. Resistance change was measured by painting conductive silver epoxy across the negative embossed electrode interface and wrapping copper tape around the positive extruded interface. Ultraflexible wires were soldered to the copper tape and connected to a Source Meter (Keithley 2450). The stretching stage used a 5 kg load cell (Degraw, 050HX) and a load cell amplifier (Sparkfun, HX711) to measure stress response during displacement. Analog values were paired to a calibration curve generated by loading the cell with known weights and plotting a line of best fit for analog output to known values. Data was logged using serial communication from a microcontroller (Arduino, Arduino Mega 2360) to a personal computer using a serial monitor logger (CoolTerm). Results were discarded if slippage occurred via visual inspection and interpretation of generated waveform. Electrodes were stretched to 30% a minimum of 20 times to ensure plastic deformation was fully played out.

### Failure Mode Testing

Integration strategies were evaluated for failure response. A miniaturized functional unit for both textile-integrated and biosymbiotic systems were created. The textile-integrated functional unit consisted of an electrode in the Small Dense configuration printed directly into a compression shirt, then cut to dimensions of the biosymbiotic mesh functional unit. The textile was clamped and pulled until electrode delamination. The biosymbiotic mesh functional unit consisted of the nodal attachment from finalized mesh design and a solid infill geometry with three bolt holes cut. Electrodes were heat bonded to TPU nodes and stretched to failure.

### Electrode Integration

Electrodes were FDM printed onto a glass bed and textile-integrated via a fabric adhesive (3M). Once placed, shirts were ironed from the front (exterior) to improve adherence. Rapid textile integration for creating a fleet of shirts involved use of a double-sided fabric adhesive (3M) bonded to the shirt-interfacing side of the electrode. Once the centroid of electrode and desired shirt placement were matched (visual inspection, manual pen markings for placement deviations), a small hole 2 cm in diameter was created in the shirt allowing for signal pass-through and interface with downstream electronics. Fabric adhesive and electrode were fused to the textile platform by carefully ironing the composite from the shirt exterior.

### Electrode Placement for ECG

For male subjects, pectoral electrode midlines were initially placed at an x-distance of 9 cm from midline and 7 cm from acromion. Thoracic electrodes were placed an x-distance of 11 cm from midline and y-distance 14 cm from axilla. Female subjects saw pectoral electrode midlines placed at an x-distance of 8 cm from midline and 7 cm from acromion. Thoracic electrodes were placed an x-distance of 10.5 cm from midline and y-distance 13 cm from axilla. Individualized placements showed slight deviations (maximum 2 cm) from these values, using visual inspection to maximize contact area.

### Electrode Characterization Experiments

### Firmware Parameters

Data collected for these experiments was made using MAX30001EVSYS with the following settings:

ECG settings: Channel Gain: 20V/V. Sample Rate: 128 sps. Digital LPF cutoff: 40.96 Hz. Digital HPF Cutoff: 0.5 Hz. Resistive Bias Value: 100 MOhm

BioZ settings: Current Magnitude: 32 uA. Current Driver Frequency: 80 KHz. Mode: Chopped w/o LPF. Channel Gain: 10V/V. Sample Rate: 64 sps. Digital LPF Cutoff: 4 Hz. Digital HPF Cutoff: Bypass

### Peak-Peak ECG Characterization

Each biosymbiotic electrode geometry was FDM printed directly into compression shirt textile. Electrodes were cut out of textile-embedded platform and had Velcro adhered. A non-integrated shirt was created with female Velcro adhered in prescribed ideal electrode placements as described above. ECG waveforms were collected from each electrode geometry and gold standard Ag/AgCl electrode from a development board (MAXIM, MAX30001EVSYS). Pak amplitudes were obtained by isolating each heartbeat's R and S points and adding the absolute value of the two. R peaks were located via the findpeaks function in MATLAB, with minimum peak distance of 0.3593 seconds (167 bpm) and peak threshold of 0.4 mV. S peaks were located with the same process, but an inverted dataset as this function cannot detect negative peaks. These parameters are necessary, as they exclude confounding measurements from detection of non-relevant features such as P and T waves. Ten consecutive heartbeats per electrode geometry after settling were chosen for evaluation.

### ECG Waveform Correlation Experiment

ECG waveforms were obtained from a subject at rest, standing on two feet, with wired connections taped to the subject to reduce 1 Hz motion artifacting noise. Computers and development kits used for collecting data sat on ground planes made of plastic (PLA) and copper and were not plugged into a wall outlet, reducing 50-60 Hz noise artifacts. Correlation coefficients were obtained via MATLAB's corrcoef function, following use of the following equation:

$$ρ ( A , B ) = \frac{1}{N - 1} {\sum }_{i = 1} \frac{{A}_{i} - {μ}_{A}}{{σ}_{a}} * \frac{{B}_{i} - {μ}_{B}}{{σ}_{b}}$$

Where A represents generated dry electrode data, B represents generated gold standard gel electrode data, μ represents sample mean, and σ represents signal SD. As data was collected simultaneously, data was timesynced, and raw ECG voltage readings were compared point-by-point across thirty consecutive heartbeats to generate an R value.

### Electrical Impedance Pneumography Tidal Breathing Amplitude Experiment

Determination of highest peak amplitude for inspiration via impedance pneumography used the same Velcro platform as the previous tests. Five consecutive maximal inspirations and expirations were recorded, corresponding peaks aggregated and mean maximal expiration was subtracted by mean maximal inspiration.

### Bioimpedance Performance Concerning Current Injection Frequency

In this experiment, LA and RA electrodes were placed as in ECG trials using the Large Dense configuration. The current drive frequency settings available to the analog front-end chip (Maxim, MAX30001) were each tested for maximal peak-peak impedance. Current drive frequency values are: 125 Hz, 250 Hz, 500 Hz, 1 kHz, 2 kHz, 4 kHz, 8 kHz, 18 kHz, 40 KHz, 80 KHz, and 128 kHz. Each frequency was evaluated for its effect on the BioZ readings of the Large Dense electrode through the following procedure: The subject's breath was held with full lungs for the first five seconds of the experiment, followed by five full breaths out and five full breaths in. At the top of the fifth breath, the subject held their breath for 15 s. At the end of 15 s, the subject released their breath and took four more complete breaths in and out. Plotted values represent average change from maximal expiration to inspiration.

### Electrical Impedance Myography Characterization

EIM testing was performed asynchronously across electrode types. A biosymbiotic mesh platform with modular interface for each electrode was fabricated to ensure uniform contact pressures and electrode placements. Electrodes were placed with midlines on the proximal belly (12.5 cm from olecranon) of the brachialis of the right forearm and midline of the distal brachialis (10 cm from radial bump on wrist). The MAX30001 development kit was used with default BioZ settings selected. A hand dynamometer (Sutekus) was squeezed to a stable reading of 15 kg, held for 5 seconds, relaxed for 10 seconds, and repeated four times. Change in BioZ was measured as amplitude change from the average reading of the final five seconds of stable baseline to the average reading of the five seconds corresponding to 15 kg squeeze. Standard Deviation was obtained similarly. Signal-to-Noise ratio was obtained from the same test. Noise was defined as the deviation of signal from mean reading when no additional muscle signal was present. Signal was defined as deviation from the mean of muscle contraction readings. Signal to noise ratio was obtained by dividing each signal mean by its preceding baseline noise.

### Motion Artifact in ECG Characterization

Calculation of Root Mean Square Error and self-correlation during a walk test used gold standard Ag/AgCl electrodes and a stainless steel (SS) cutout of gold standard dry electrode geometry (45×19 mm) used in chest-strap based activity trackers (OmegaWave) were compared in typical and extreme motion artifacting scenarios. The MAX30001EVSYS was used to attach to each electrode type via wires which were banana clipped to the electrode. Two electrodes of each type were placed in the intercostal space between the fourth and fifth rib of a subject, equidistant from sternum. A cloth band was wrapped over these electrodes to standardize contact pressure and ensure a uniform contact area. Gel electrodes were adhered to skin and placed under the same cloth chest band. SS electrodes had a small weight corresponding to sensor battery pack weight added. The subject performed ten seconds of rest followed by 25 seconds of walking on a treadmill at 2.0 mph three times consecutively. Root Mean Square Error was calculated by creating a window of 0.7 s, centered at the R-peak of each heartbeat. Each waveform was overlaid and a smoothing spline for each electrode type was generated via MATLAB's Curve Fitting Tool. Quality metrics including RMSE and signal correlation (R²) are generated automatically.

### Extreme Motion in ECG Characterization

Motion artifact amplitude and settling time were obtained by adhering electrodes to skin in the same manner as described in the previous section. The subject stood still on a 15 cm tall box for 5 seconds to obtain a stable ECG reading, then jumped off the box onto the floor. The subject waited for 10 seconds, allowing signal to restabilize, stepped back onto the box, waited 5 more seconds and repeated four times. This test was performed for each electrode three times for a total of 15 artifacts analyzed. Visual inspection determined amplitude size, where the sum of largest deviation in total magnitude from stable signal was evaluated. R peaks were excluded from this analysis. Settling time was also determined by visual inspection by noting the location signal deviated from stable ECG readings to the point where feature identification was again possible.

### Electrode Performance Vs Gel Electrode Over Indefinite Periods (ECG)

The Large Dense Electrode configuration was chosen. A biosymbiotic textile-integrated system was created with each electrode printed directly into it. Two gel electrode brands were adhered to the subject's skin around each gel electrode. Each electrode was placed 5 cm from biosymbiotic electrode midline, with uniform directionality for each electrode brand (i.e., all 3M electrodes were placed directly superior to a biosymbiotic electrode, all MediTrace electrodes were placed directly medial to a biosymbiotic electrode, etc.). Three times daily, 60 seconds of continuous ECG was taken asynchronously from each electrode type using wired connections to the MAX30001EVSYS. The average R peak amplitude was added to the average absolute value S peak amplitude for each dataset, creating a mean P-P amplitude for that dataset. The data was normalized by dividing the complete array of R to S P-P amplitudes by the mean generated previously. Each day this process was repeated to generate the results shown in FIG. 4b. Measurements continued until gel electrodes could no longer adhere to skin. No special skin preparation, including shaving of hair or debriding the epidermis was undertaken. This shirt was worn continuously apart from washing in a delicate cycle in the user's household washing machine (Samsung) every two days and air dried overnight. The subject was permitted to wash the shirt more often if hygiene concerns necessitated it.

### Chronic Electrode Stability and Skin-Electrode Impedance

Contact impedance over a 160-hour timeframe was determined by comparison of four gel electrode brands with the biosymbiotic electrode platform. Impedance pneumography measurements were obtained via the MAXIM30001EVSYS BioZ default setting (current magnitude 32 uA, driver frequency 80 kHz). Obtained values represent the average Z value of a twenty-second breath hold, representing a stable physiological skin-electrode bioimpedance reading. Measurements were taken every twelve hours at minimum. The experiment concluded when the final gel electrode completely detached from the subject's skin.

### Fully Integrated Wearable Testing

A Raspberry Pi (Raspberry Pi; 4 Model B) was configured to search for the electrophysiology device and continuously log advertising characteristics of the ECG and BioZ modules using a custom Python script at start. This data was logged onto an external hard drive, then imported into MATLAB for further analysis.

### Multiple Subject, Multiple Activity Direct Comparison to Activity Tracker Gold Standard

Comparison to gold standard experiments utilized the fully realized textile-integrated biosymbiotic system and involved four subjects performing six total instances of tasks ranging from 10 to 45 minutes in activities such as: running up and down stairs, stationary biking, strength training, and rowing machine. Two male subjects (190.5 cm, 88.9 kg and 187.6 cm, 92 kg), and two female subjects (167.6 cm, 58.9 kg and 172.7 cm, 68 kg) wore a textile-integrated biosymbiotic platform with slight modifications to electrode placement for electrode conformality. The Polar H10 chest strap was placed with midline centered at the sternum, passing through the 4th and 5th rib intercostal space. Activities were timestamped and continuous ECG was recorded. HR was obtained in post-processing by using the findpeaks function on raw data obtained from the biosymbiotic device, targeting the R peaks in the desired timeframe. Heart rate was calculated by dividing 60 by each R-R peak interval. As the Polar H10 samples once per second, and HR is calculated in postprocessing based on the variable heart rate, both generated datasets were upsampled to 2 Hz for comparison and Bland-Altman analysis. Upsampled heart rate data from the biosymbiotic device was low-pass filtered at 1 Hz. Bland-Altman and Coefficient of Variance plots were generated in MATLAB by using input parameters of upsampled biosymbiotic HR values and upsampled Polar H10 HR values with the BlandAltman.m function open-sourced by Ran Klein of The Ottawa Hospital.

### Full-Day and Sleep Recording Data Acquisition

Chronic ECG performance was evaluated using the fully realized textile-integrated biosymbiotic device. As hygienic concerns preclude an individual from wearing a textile-integrated platform for indefinite periods of time on end, a typical use case of a full day of regular activity and a night of sleep were selected for continuous performance analysis. A male subject (190.5 cm, 88.9 kg) wore a shirt for 25 continuous hours through key typical daily activities such as a workday, physical cardiovascular activity, and sleep. The Bluetooth module experienced data dropout at varied points in the testing cycle, and this data was excluded. A female subject (167.6 cm, 58.9 kg) wore a shirt for 9 continuous hours through her typical sleep cycle. SNR values were calculated and condensed for visualization purposes by averaging SNR over 20-minute intervals beginning from experiment, leaving 3 data points per hour on the shown graphs.

### Electrical Impedance Myography Performance Over Chronic Timescales

Chronic muscle bioimpedance experiments were performed utilizing the fully realized biosymbiotic mesh-integrated system. A biosymbiotic mesh was designed such that electrodes fell with midlines on the proximal belly (12.5 cm from olecranon) of the brachialis of the left forearm and midline of the distal brachialis (10 cm from radial bump on wrist). Each morning, the subject properly warmed up forearm flexors to comfort and then performed a maximal contraction using a hand dynamometer (Sutekus). Maximal contraction values were recorded and used to generate a performance curve for that day. The subject calculated a loading regime that matched 80% to 10%, descending by 10%, of that daily 1 RM and was instructed to perform three hand dynamometer holds matching each prescribed intensity for five seconds each. This protocol was repeated twice more on the same day (roughly corresponding to mealtimes), and a new daily 1 RM was established for the following day the next morning. An example loading protocol is shown in FIG. 16. The TPU-integrated mesh remained on the subject's forearm for the duration of the 11-day experiment.

### Example Fabrication Methods

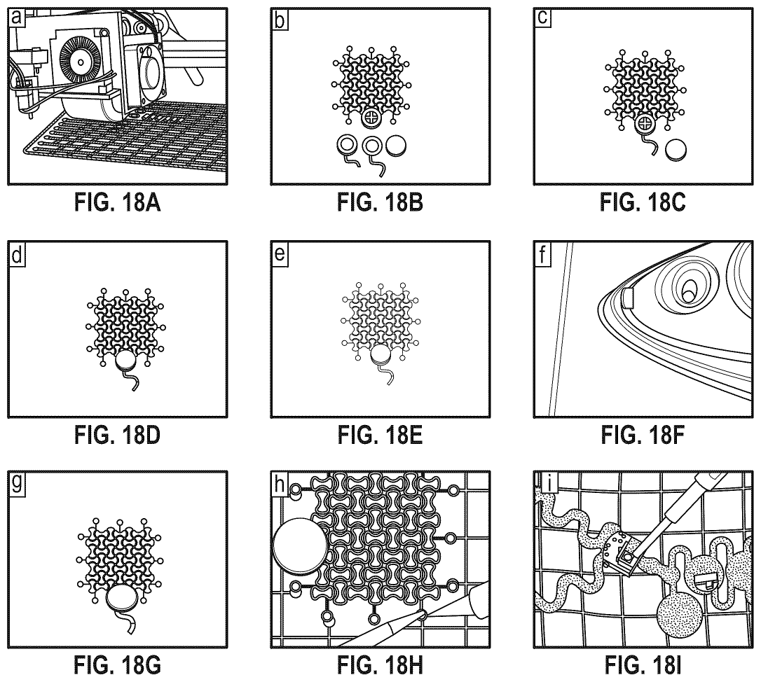

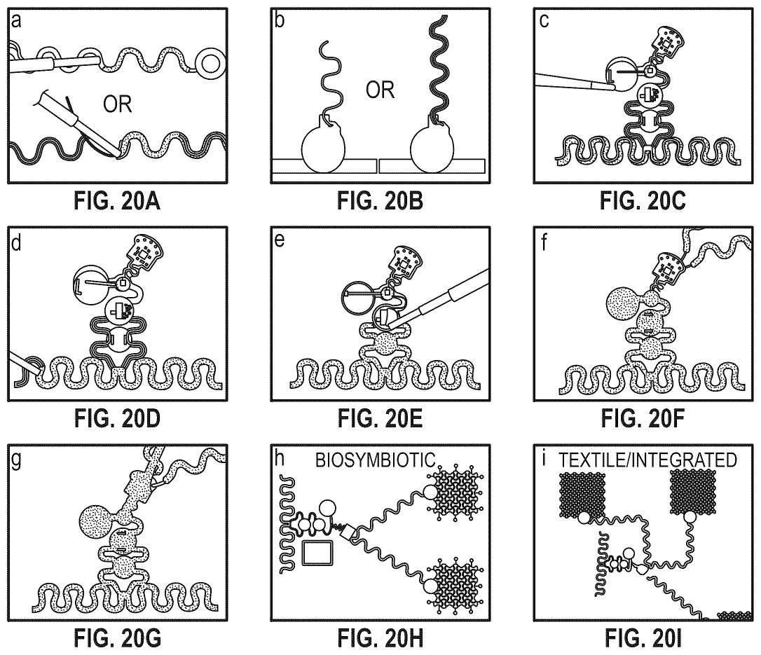

FIGS. 18A-18I illustrate example fabrication steps of a wearable biosymbiotic electrophysiology system according to the teachings of the present disclosure; where FIG. 18A illustrates FDM printing nonconductive TPU biosymbiotic mesh; FIGS. 18B, C, and D illustrate visualization of assembly of the conductivity elements to an electrode mesh; FIGS. 18E and F illustrate parchment paper overlay for heat-bonding with an iron; FIG. 18G is a photo of the electrode assembly; FIG. 18H illustrates heat bonding of the electrode with the biosymbiotic mesh platform; and FIG. 18I illustrates integration with electronics using the steps illustrated in reference to FIG. 20 (described below). Details of the process steps of FIGS. 18A-18I are described below.

2D mesh drawings were exported from AutoCAD and imported to 3D modeling software (Autodesk, Fusion 360) for extrusion. These models were exported as Stereolithography (STL) files and imported into a 3D slicing software (Prusa3D, PrusaSlicer) which converts the model into machine code usable by a 3D printer. A fusion depositing modeling printer (Creality, CR-10S) was retrofitted to use an all-metal direct drive extruder (Creality, Sprite) with an automatic bedleveling unit (Antclabs, BLTouch). TPU (NinjaTek, NinjaFlex) was printed at 45 mm-1 s at an extruder temperature of 225C and bed temperature of 45C, as shown in FIG. 18A.

Biosymbiotic electrodes integrate conductive elements on the benchtop prior to system integration, with sequential assembly steps detailed in FIGS. 18B-D. Specifically, parchment paper was laid over the top and heat-pressed with a household iron set to high, shown in FIG. 18 E and F, with the completed assembly shown in FIG. 18G. Biosymbiotic electrodes used in this system feature circular connection nodes of 5 mm diameter, which interface with a corresponding opening in the nonconductive TPU attached by heat bonding, shown in FIG. 18H These electrodes interface with electronics through wire or a FlexPCB, which was encapsulated using UV-curable flexible resin and soldered to the biopotential electronic node, which was subsequently encapsulated as shown in FIG. 18I. Chronic device segments were joined by melting circular nodes of 5 mm diameter together. Short-term use-case biosymbiotic systems were outfitted with clinging adhesive (Velcro) for rapid donning and doffing of the device.

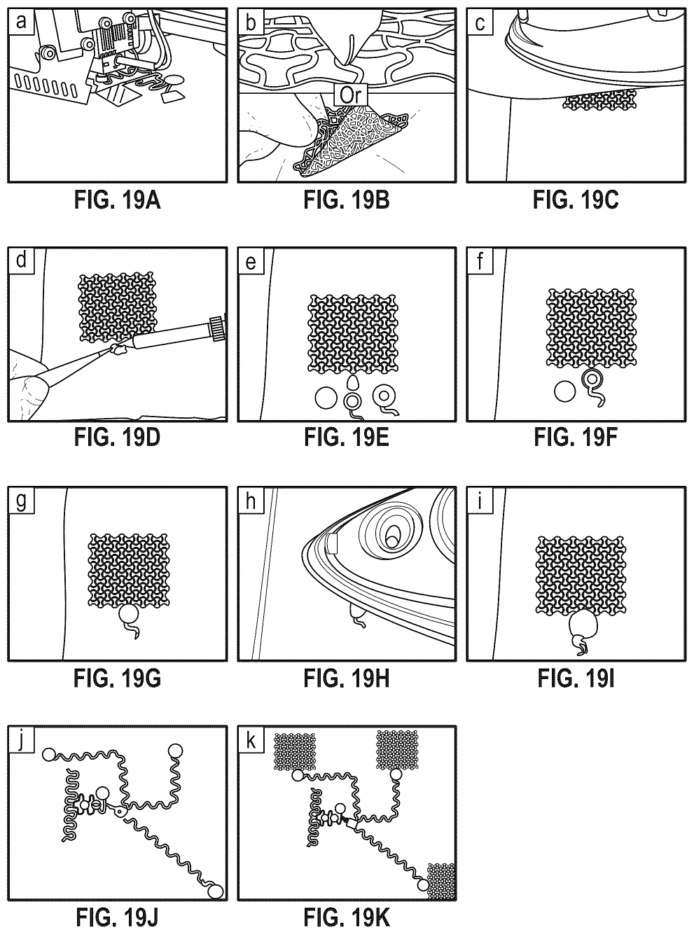

FIGS. 19A-19K illustrate example fabrication steps for the textile-integrated electrophysiology systems according to the teachings of the present disclosure; where: FIG. 19A illustrates FDM printing nonconductive TPU device housing into the textile; FIG. 19B illustrates integrating biosymbiotic electrodes via direct printing (top), or fabric adhesive (bottom); FIG. 19C illustrates parchment paper overlay for heat-bonding using an iron; FIG. 19D illustrates creating a through-hole in the textile for electronic integration; FIGS. 19E, F, and G visualization of assembly of the conductivity elements to an electrode in association with the textile; FIG. 19H illustrates parchment paper overlay for heat-bonding using an iron; FIG. 19I is a photo of the assembly; and FIGS. 19J, and K illustrate integration with electronics using the steps illustrated in reference to FIGS. 20A-20I (described below). Details of the process steps of FIGS. 19A-19K are described below.

The device housing for biopotential electronics was first FDM printed directly into the textile, oriented at x and y midline of a subject's sternum, shown in FIG. 19A. Electrodes were FDM printed either directly into the textile or onto a glass bed and later textile-integrated via a fabric adhesive (Aleene's Original Fabric Fusion), shown in FIG. 19B. Fabric adhesive and electrode were fused to the textile by ironing from the shirt exterior, shown in FIG. 19C. Once the centroid of the electrode and desired shirt placement were matched, a small hole 2 cm in diameter was created in the shirt allowing for signal pass-through and interface with electronics, shown in FIG. 19G. The 3D printed serpentine electrodes interact with downstream electronics via a conductive Pyralux double-sided copper clad laminate (AG185010RY; constituent layers, 18 μm copper, 50 μm polyimide, and 18 μm copper) FlexPCB laser-cut serpentine element with a 4 mm² circular opening press-fit into a raised “male” circular pillar at the electrode x-midline and y-minima. This serpentine element was paired with a “cap and channel,” represented by a conductive 3D printed element that was a negative of the FlexPCB, and a printed cap that fits atop the circular pillar, also a press fit. The electrode was assembled such that the circular pillar of the electrode body intersects with the channel component, FlexPCB was carefully inserted, and the cap was placed on top, creating a multi-layered structure of 2.2 mm in height. This complete assembly is shown in FIG. 19 E-G. Once the desired Flex-PCB orientation was obtained, the four layers were heat-bonded. A layer of parchment paper was laid on top of the assembled electrode to avoid adhesion to the iron as shown in FIG. 19H, with post-bonded height of 1.6 mm (FIG. 19I). A schematic of this integration strategy is shown in FIG. 7B. Wire or FlexPCB were placed into nonconductive FDM printed TPU serpentine elements adhered to the textile, then encapsulated in flexible UV resin. Electrical connections were soldered as shown in FIG. 19J. The complete device was then encapsulated in flexible UV resin, shown in FIG. 19K.

FIGS. 20A-20I illustrate example steps for electronics fabrication for biosymbiotic and textile-integrated electrophysiology systems according to the teachings of the present disclosure, where; FIG. 20A illustrates disposing FlexPCB/Wire into nonconductive TPU serpentine housing and encapsulating; FIG. 20B illustrates soldering wire to FlexPCB component of electrode, if needed; FIG. 20C illustrates placing the device in a nonconductive TPU housing; FIGS. 20D, and E illustrate beginning to encapsulate using UV curable white flexible resin; FIG. 20F illustrates attaching wire/FlexPCB to biopotential breakout pins G) complete encapsulation H) photographic images of completed biosymbiotic and textile-integrated systems. Details of the process steps of FIGS. 20A-20I are described below.

The connection was accomplished via printed serpentine channels of 0.45 mm wide×3 mm wide nonconductive TPU. FlexPCB or ultraflexible wire (Calmont, 36 gauge) was woven into the TPU serpentines, then encapsulated with UV-curable flexible resin (Superfast Superflex) of shore hardness 80A, shown in FIG. 20A. FIG. 20B shows electronic connections extending from a complete electrode assembly. Electronics, as described in FIG. 1 integrate with biosymbiotic and textile-integrated systems by an FDM printed nonconductive thermoplastic polyurethane (TPU) (NinjaTek, NinjaFlex) of shore hardness 85A in a 0.45 mm thick device housing with 0.2 mm walls printed directly into textile and biosymbiotic systems. The device housing features a. 1 mm offset to fit the assembled device footprint. The fully assembled flexible electrophysiology device shown in (FIG. 20C) was carefully placed into the device housing and encapsulated with UV-curable (SuperFast Superflex) white flexible resin, shown in FIGS. 20C-G. Completely assembled devices are shown in FIG. 20H.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

“Circuit” and “Circuitry”, as used in any embodiment herein, may comprise, for example, singly or in any combination, hardwired circuitry, programmable circuitry such as processors comprising one or more individual instruction processing cores, state machine circuitry, and/or firmware that stores instructions executed by programmable circuitry, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), application-specific integrated circuit (ASIC), programmable logic devices (PLD), digital signal processors (DSP), field programmable gate array (FPGA), logic gates, registers, semiconductor device, chips, microchips, chip sets, etc.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

What is claimed:

1. A sensor system for measuring biosignals, comprising:
an electrode to detect a biosignal, the electrode being formed of an electrically conductive mesh structure comprising a plurality of serpentine filaments forming defining mesh openings therebetween;
electrical conductivity elements electrically coupled to the electrode; and
biosignal acquisition circuitry electrically coupled to the electrical conductivity elements to receive the biosignal, to process the biosignal, and to harvest radio frequency energy to power the circuitry and to obtain measurements of the biosignal.

2. The sensor system of claim 1, wherein the electrode is formed of a flexible carbon-doped thermopolyurethane (TPU).

3. The sensor system of claim 1, wherein the mesh size of the electrode is selected based on a selected biosignal for sensing.

4. The sensor of claim 1, wherein the biosignal is selected from one or more of electrocardiogram (ECG), Electrical Impedance Pneumography (EIP), and Electrical Impedance Myography (EIM).

5. The sensor of claim 1, wherein the electrode further comprising a coupling pad having a crown portion.

6. The sensor of claim 5, wherein the electrical conductivity elements include:
a conductive thermopolyurethane (TPU) member having a ring-shaped coupling member defining a first opening and serpentine channel member extending from the coupling member;
a flexible metallic member having a metallic ring-shaped member defining a second opening and dimensioned to be received within the ring shaped coupling member, and a serpentine metallic member extending from the metallic ring-shaped member dimensioned to be received in the channel member; and
a cap member configured to be received into the first and second openings and to engage the crown portion of the coupling pad.

7. The biosymbiotic sensor system of claim 1, wherein the electrode is 3-D printed.

8. The biosymbiotic sensor system of claim 1, wherein the electrode, the electrical conductivity elements, and the biosignal acquisition circuitry are integrated into a wearable mesh.

9. The biosymbiotic sensor system of claim 1, wherein the electrode, the electrical conductivity elements, and the biosignal acquisition circuitry are integrated into a textile.

10. A biosymbiotic sensor system integrated into a textile, comprising:
an electrode to detect a biosignal, the electrode being formed of an electrically conductive mesh structure comprising a plurality of serpentine filaments forming defining mesh openings therebetween; the electrode being disposed on an inside surface of the textile to contact skin to receive the biosignal from the skin;
electrical conductivity elements electrically coupled to the electrode; the electrical conductivity elements being disposed on an outside surface of the textile; and
biosignal acquisition circuitry electrically coupled to the electrical conductivity elements to receive the biosignal, to process the biosignal, and to harvest radio frequency energy to power the circuitry and to obtain measurements of the biosignal, the biosignal acquisition circuitry being disposed on the outside surface of the electrode.

11. The sensor system of claim 10, wherein the electrode is 3D printed using a flexible carbon-doped thermopolyurethane (TPU) material.

12. The sensor system of claim 10, wherein the mesh size of the electrode is selected based on a selected biosignal for sensing; and wherein the biosignal is selected from one or more of electrocardiogram (ECG), Electrical Impedance Pneumography (EIP) and Electrical Impedance Myography (EIM).

13. The sensor of claim 10, wherein the electrode further comprising a coupling pad having a crown portion; and wherein the electrical conductivity elements include:
a conductive thermopolyurethane (TPU) member having a ring-shaped coupling member defining a first opening and serpentine channel member extending from the coupling member;
a flexible metallic member having a metallic ring-shaped member defining a second opening and dimensioned to be received within the ring shaped coupling member, and a serpentine metallic member extending from the metallic ring-shaped member dimensioned to be received in the channel member; and
a cap member configured to be received into the first and second openings and to engage the crown portion of the coupling pad through an opening in the textile.

14. A wearable biosymbiotic sensor system, comprising:
flexible mesh structure dimensioned to be formed around a selected anatomical region;
an electrode to detect a biosignal, the electrode being formed of an electrically conductive mesh structure comprising a plurality of serpentine filaments forming defining mesh openings therebetween; the electrode being disposed within opening of the flexible mesh structure to contact skin to receive the biosignal from the skin;
electrical conductivity elements electrically coupled to the electrode; the electrical conductivity elements being disposed on the flexible mesh structure; and
biosignal acquisition circuitry electrically coupled to the electrical conductivity elements to receive the biosignal, to process the biosignal, and to harvest radio frequency energy to power the circuitry and to obtain measurements of the biosignal, the biosignal acquisition circuitry being disposed on the flexible mesh structure.

15. The wearable biosymbiotic sensor system of claim 14, wherein the electrode is 3D printed using a flexible carbon-doped thermopolyurethane (TPU) material.

16. The wearable biosymbiotic sensor system of claim 14, wherein the mesh size of the electrode is selected based on a selected biosignal for sensing; and wherein the biosignal is selected from one or more of electrocardiogram (ECG) and Electrical Impedance Pneumography (EIP) and Electrical Impedance Myography (EIM).

17. The wearable biosymbiotic sensor of claim 14, wherein the electrode further comprising a coupling pad having a crown portion; and wherein the electrical conductivity elements include:
a conductive thermopolyurethane (TPU) member having a ring-shaped coupling member defining a first opening and serpentine channel member extending from the coupling member;
a flexible metallic member having a metallic ring-shaped member defining a second opening and dimensioned to be received within the ring shaped coupling member, and a serpentine metallic member extending from the metallic ring-shaped member dimensioned to be received in the channel member; and
a cap member configured to be received into the first and second openings and to engage the crown portion of the coupling pad through an opening in the textile.

18. The wearable biosymbiotic sensor of claim 14, wherein the electrode further comprising a plurality of bonding pads formed along the outer periphery of the electrodes, the bonding pads being formed of a material to heat fuse with the flexible mesh structure.
